SEQUENCE LISTING (12 sequences)

SEQ ID NO: 1 - DNA, 21 bp, synthetic construct
  misc_feature 1..21: note = synthetic construct
  source 1..21: mol_type = other DNA
caccaacaatggcaacatcag

SEQ ID NO: 2 - DNA, 19 bp, synthetic construct
  misc_feature 1..19: note = synthetic construct
  source 1..19: mol_type = other DNA
gtcgatgcacaactgggtg

SEQ ID NO: 3 - DNA, 21 bp, synthetic construct
  misc_feature 1..21: note = synthetic construct
  source 1..21: mol_type = other DNA
gcctatgtctcagcctcttct

SEQ ID NO: 4 - DNA, 21 bp, synthetic construct
  misc_feature 1..21: note = synthetic construct
  source 1..21: mol_type = other DNA
tctgggccatagaactgatga

SEQ ID NO: 5 - DNA, 20 bp, synthetic construct
  misc_feature 1..20: note = synthetic construct
  source 1..20: mol_type = other DNA
cgctgttcttttcctcttgg

SEQ ID NO: 6 - DNA, 20 bp, synthetic construct
  misc_feature 1..20: note = synthetic construct
  source 1..20: mol_type = other DNA
agtccggatctaggcaggtt

SEQ ID NO: 7 - DNA, 21 bp, synthetic construct
  misc_feature 1..21: note = synthetic construct
  source 1..21: mol_type = other DNA
ccaagtgctgccgtcattttc

SEQ ID NO: 8 - DNA, 21 bp, synthetic construct
  misc_feature 1..21: note = synthetic construct
  source 1..21: mol_type = other DNA
ggctcgcagggatgatttcaa

SEQ ID NO: 9 - DNA, 20 bp, synthetic construct
  misc_feature 1..20: note = synthetic construct
  source 1..20: mol_type = other DNA
agtaacggctgcgacaaagt

SEQ ID NO: 10 - DNA, 20 bp, synthetic construct
  misc_feature 1..20: note = synthetic construct
  source 1..20: mol_type = other DNA
gtcagacgttcccaggatgt

SEQ ID NO: 11 - DNA, 20 bp, synthetic construct
  misc_feature 1..20: note = synthetic construct
  source 1..20: mol_type = other DNA
acagcttctttgcagctcct

SEQ ID NO: 12 - DNA, 17 bp, synthetic construct
  misc_feature 1..17: note = syntehtic construct
  source 1..17: mol_type = other DNA
atacagcccggggagca

(19) United States
(12) Patent Application Publication
(10) Pub. No.: US 2024/0000741 A1
(43) Pub. Date: Jan. 4, 2024

(54) ALL-TRANS RETINOIC ACID ENHANCES RADIOTHERAPY AND OVERCOMES IMMUNE SUPPRESSION FOR CANCER THERAPY

(71) Applicant: The University of Chicago, Chicago, IL (US)

(72) Inventors: Wenbin Lin, Chicago, IL (US); Ralph R. Weichselbaum, Chicago, IL (US); Hua Liang, Chicago, IL (US); Yingying Li, Chicago, IL (US)

(21) Appl. No.: 18/329,289

(22) Filed: Jun. 5, 2023

Related U.S. Application Data
(60) Provisional application No. 63/348,745, filed on Jun. 3, 2022.

(51) Int. Cl. A61K 31/203; A61K 39/395; A61P 35/00

(52) U.S. Cl. CPC A61K 31/203; A61K 39/3955; A61P 35/00

(57) ABSTRACT

Methods of treating cancer comprising the use of combinations of a retinoid, e.g., all-trans retinoic acid (ATRA), and radiotherapy are described. The administration of a retinoid can enhance the effect of radiotherapy, including enhancing fractionated, low-dose radiotherapy. Use of the combination increases tumor necrosis factor alpha (TNF-α)- and inducible nitric oxide synthase (iNOS)-producing inflammatory macrophages in local (radiated) and distal (non-radiated) tumors. The methods can optionally further involve the use of checkpoint blockade immunotherapy.

### CROSS-REFERENCE TO RELATED APPLICATIONS

This application claims priority to and benefit of U.S. Provisional Patent Application Ser. No. 63/348,745, filed Jun. 3, 2022, the disclosure of which is incorporated herein by reference in its entirety.

### STATEMENT OF GOVERNMENT SUPPORT

This invention was made with government support under CA195075, and CA253655 awarded by the National Institutes of Health. The government has certain rights in the invention.

### REFERENCE TO SEQUENCE LISTING XML SUBMITTED ELECTRONICALLY

The content of the Sequence Listing XML filed using Patent Center as an XML file (Name: 3072_23_2. xml; Size: 16,091 bytes; and Date of Creation: Jun. 2, 2023) is incorporated herein by reference in its entirety.

### TECHNICAL FIELD

The presently disclosed subject matter relates to methods of treating cancer, including solid tumors, using a combination of administration of a retinoid, such as all-trans retinoic acid (ATRA), and radiotherapy using ionizing irradiation. The methods can further comprise the use of checkpoint blockade immunotherapy. The use of the retinoid can enhance the ability of radiotherapy to treat cancer, thereby providing for the effective use of fractionated, low dose radiotherapy.

### ABBREVIATIONS

° C.=degrees Celsius
%=percentage
μg=microgram
μM=micromolar
ACT=adoptive T cell therapy
ATRA (or RA)=all-trans retinoic acid
BMDCs=bone marrow derived dendritic cells
cGy=centigray
cm=centimeter
CTLA-4=cytotoxic T lymphocyte-associated protein 4
DAMP=damage-associated molecular pattern
DC=dendritic cell
Gy=gray
IFN-γ=interferon-gamma
IL=interleukin
Inf-MAC=inflammatory macrophages
iNOS=inducible nitric oxide synthase
IO=immune-oncology
IR=ionizing radiation
kg=kilogram
KO=knock out
kVp=peak kilovoltage
mA=milliampere
MDSC=myeloid-derived suppressor cell
mg=milligram
ml=milliliter
mm=millimeter
mmol=millimole
nM=nanomolar
NO=nitric oxide
N.S.=not significant
PD-L1=programmed death ligand 1
PBS=phosphate buffered saline
RT=radiotherapy
SD=standard deviation
SEM=standard error of the mean
TME=tumor microenvironment
TNF-α=tumor necrosis factor-alpha
T_{reg}=regulatory T cells
WT=wild type

### BACKGROUND

Chemotherapy and radiotherapy are among the powerful anticancer therapies used across a multitude of cancer types. However, the effective use of these therapies can be limited due to undesirable side effects to healthy cells at high dosages. In addition, these therapies are often compromised by the development of drug and radio-resistance by tumor cells.

Accordingly, there is an ongoing need for additional cancer treatment methods and compositions, such as those with enhanced anticancer efficacy. For instance, there is an ongoing need for additional cancer treatment methods and compositions that can be used to treat drug- and/or radio-resistant cancers and/or that can be used to treat cancer while avoiding the development of drug- and/or radio-resistant cancer cells.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a method for treating a cancer in a subject in need thereof, the method comprising: administering to the subject a retinoid or a pharmaceutically acceptable salt thereof; and exposing at least a portion of the subject to ionizing irradiation energy.

In some embodiments, the retinoid is selected from the group comprising retinol, retinal, a retinoic acid, an ester or amide of a retinoic acid, a metabolite of a retinoic acid, and mixtures thereof. In some embodiments, the retinoid is selected from the group comprising all-trans retinoic acid (ATRA), 9-cis-retinoic acid, 13-cis retinoic acid, fenretinide, retinal, 4-hydroxy-retinoic acid, 4-oxo-retinoic acid, 18-hydroxy-retinoic acid, 5,6-epoxy-retinoic acid, and mixtures thereof. In some embodiments, the retinoid comprises or consists of ATRA.

In some embodiments, the cancer is a solid tumor cancer. In some embodiments, the cancer is selected from the group comprising a skin cancer, a connective tissue cancer, an adipose cancer, a breast cancer, a head and neck cancer, a lung cancer, a stomach cancer, a pancreatic cancer, an ovarian cancer, a cervical cancer, a uterine cancer, an anogenital cancer, a kidney cancer, a bladder cancer, a colon cancer, a prostate cancer, a central nervous system (CNS) cancer, a retinal cancer, a neuroblastoma, and a lymphoid cancer, optionally wherein the cancer is a colon cancer or a kidney cancer.

In some embodiments, the method further comprises administering to the subject an additional therapeutic agent or treatment. In some embodiments, the additional therapeutic agent or treatment is selected from an immunotherapy agent and/or a cancer treatment, wherein the cancer treatment is selected from the group consisting of surgery, chemotherapy, toxin therapy, cryotherapy and gene therapy. In some embodiments, the additional therapeutic agent or treatment comprises an immunotherapy agent. In some embodiments, the immunotherapy agent is an immune checkpoint inhibitor. In some embodiments, the immune checkpoint inhibitor is selected from the group comprising a PD-1 inhibitor, a PD-L1 inhibitor, a CTLA-4 inhibitor, an IDO inhibitor, a CCR7 inhibitor, an OX40 inhibitor, a TIM3 inhibitor, and a LAG3 inhibitor, optionally wherein the immune checkpoint inhibitor is a PD-L1 inhibitor.

In some embodiments, the retinoid is administered orally. In some embodiments, the exposing is performed by exposing said at least a portion of the subject to a fraction of a total dose of ionizing irradiation energy on two or more separate days until said at least a portion of the subject is exposed to said total dose of ionizing irradiation energy, optionally wherein said two or more separate days are two or more consecutive days.

In some embodiments, a combination of the administering and the exposing provides enhanced tumor growth control compared to a treatment comprising the administering alone or the exposing alone. In some embodiments, the combination of the administering and the exposing provides enhanced tumor growth control for a tumor not directly targeted by said administering and/or said exposing. In some embodiments, a combination of the administering and the exposing provides enhanced or comparable tumor growth control using a lower total dose of ionizing radiation energy compared to a treatment consisting of exposing the subject to ionizing radiation alone. In some embodiments, a combination of the administering and the exposing provides an increase in inducible nitric oxide synthase (iNOS)-producing myeloid cells in the subject. In some embodiments, the combination provides an increased level of CD11b+iNOS+ cells in a tumor in the subject. In some embodiments, a combination of the administering and the exposing provides an increase in tumor necrosis factor-alpha (TNF-α)-producing myeloid cells in the subject. In some embodiments, a combination of the administering and the exposing provides protection from tumor recurrence.

Accordingly, it is an object of the presently disclosed subject matter to provide methods of treating cancer via combinations of a retinoid, e.g., all-trans retinoic acid (ATRA or RA), and ionizing irradiation energy, optionally in combination with immunotherapy.

These and other objects are achieved in whole or in part in the presently disclosed subject matter. An object of the presently disclosed subject matter having been stated above, other objects and advantages will become apparent upon a review of the following descriptions, examples, and figures.

### BRIEF DESCRIPTION OF THE DRAWINGS

FIGS. 1A-1G: Combining local ablative ionizing radiation (IR) and all-trans retinoic acid (ATRA) inhibited tumor growth and acquired antitumor memory. FIG. 1A is a schematic diagram showing the treatment schedule. Daily administration of ATRA by gavage at 400 micrograms per dose (μg/dose). FIG. 1B is a graph showing a growth curve of murine colon adenocarcinoma (MC38) tumors treated with oil (filled triangles), all-trans retinoic acid (RA, filled squares), 15 gray (Gy)+oil (unfilled triangles) or 15 Gy plus RA (unfilled squares). Tumor volume is provided in cubic millimeters (mm³) as a function of time (number of days after tumor inoculation). FIG. 1C is a graph showing the percentage (%) of tumor bearing mice over time post indicated treatment (i.e., oil (filled triangles), all-trans retinoic acid (RA, filled squares), 15 gray (Gy)+oil (unfilled triangles) or 15 Gy plus RA (unfilled squares)). Animals were pooled from three separate experiments. FIG. 1D is a graph showing the survival curves of mice described in FIG. 1C. Data from three independent experiments were pooled. FIG. 1E is a graph of tumor growth curves (tumor volume in mm³ versus days after tumor inoculation) in cured (MC38) and naive mice which were re-challenged with 5×10⁶ MC38 or 1×10⁶ Lewis lung carcinoma (LLC) cells respectively. FIG. 1F is a graph showing growth curves (tumor volume in mm³ versus days after inoculation) of murine melanoma (B16) tumors in mice treated with oil (filled triangle), RA (filled square), 15Gy+oil (unfilled triangle) or 15Gy and RA combined (unfilled square). FIG. 1G is a graph showing the growth curves (tumor volume in mm³ versus days after tumor inoculation) of kidney cancer Renca tumor treated with oil (filled triangle), RA (filled square), 15Gy+oil (unfilled triangle) or 15Gy and RA combined (unfilled square). Representative data are shown from two or three experiments conducted with 5-10 mice per group, except for FIGS. 1C and 1D. Data are presented as mean±SEM. *p<0.05, **p<0.01 and ***p<0.001. N.S., not significant.

FIGS. 2A-2L: Combining local ablative ionizing radiation (IR) with all-trans retinoic acid (RA) induces inducible nitric oxide synthase (iNOS) and tumor necrosis factor-alpha (TNF-α) producing inflammatory macrophages. Murine colon adenocarcinoma (MC38) tumors were treated as indicated (oil, RA, IR+oil, or IR+RA) and harvested 4 days post start of the IR/RA treatments for analysis. FIG. 2A is a series of graphs showing a gating strategy for iNOS⁺ myeloid cells. FIG. 2B is a graph showing the frequency (as a percentage (%)) of CD11 b⁺ myeloid cells in total cells of tumors. FIG. 2C is a graph showing the percentage of major subsets of CD11b⁺ cells in tumors received IR/RA treatment. FIG. 2D is a series of graphs showing a gating strategy of iNOS⁺ inflammatory macrophages (Inf-MACs). FIG. 2E is a graph showing the percentage (%) of iNOS⁺ inflammatory macrophages in total cells of tumors. FIG. 2F is a graph showing the percentage (%) of subsets of iNOS⁺ Inf-MACs in terms of CD11c and Ly6C marker in total cells. FIG. 2G is a graph showing the concentration of iNOS product nitrite in tumor homogenate. Treated tumors were harvested on day 4 post treatment and tumor fragments were cultured in vitro. Nitrite level in culture media was measured after 2 hours. FIG. 2H is a graph showing the TNF-α⁺CD11b⁺ population (%) in tumors receiving indicated treatment. FIG. 2I is a graph showing the concentration (in picograms per milliliter (pg/ml)) of TNF-α in tumor homogenate at 4 days after start of the indicated treatments. FIG. 2J is a graph showing the percentage (%) of Inf-MACs in tumors grown in wild type (WT) or CCR2^{-/-} mice, 4 days after start of IR and RA treatment. FIG. 2K is a graph showing the tumor growth curves (tumor volume in cubic millimeters (mm³) versus days after tumor inoculation) of MC38 tumors during treatments while CD11b⁺ cell recruitment was blocked. FIG. 2L is a graph showing tumor growth curves (tumor volume in mm³ versus days after tumor inoculation) of MC38 tumors during treatments while iNOS inhibitor (1400 W) was administered. *, p<0.05; **, p<0.01; ***, p<0.001. Experiments were conducted 3 times with 3-5 mice in each group. Data in FIGS. 2K and 2L are presented as mean±SEM, the rest of the data are mean±SD. Results from representative experiments are shown.

FIGS. 3A-3L: Combining ablative ionizing radiation (IR) with all-trans retinoic acid (RA) enhances inducible nitric oxide synthase (iNOS)-dependent antitumor T cell responses. Murine colon adenocarcinoma (MC38) tumors were harvested and analyzed at day 8 after start of treatments (oil, RA, IR+oil, or IR+RA). FIG. 3A is a graph showing the percentage (%) of CD8⁺ in the total cells of tumors that received the indicated treatments. FIG. 3B is a graph showing the % of CD8⁺ IFN-γ⁺ in total cells of tumors that received the indicated treatments. FIG. 3C is a graph showing the % of CD8⁺ Granzyme 13⁺ (GzmB) in total cells of tumors that received the indicated treatments. FIG. 3D is a graph showing the % of CD4⁺ in total cells of tumors that received the indicated treatments. FIG. 3E is a graph showing the % of CD4⁺ IFN-γ⁺ in total cells of tumors that received the indicated treatments. FIG. 3F is a graph showing the % of Foxp3⁺ cells in CD4⁺ cells in tumors that received the indicated treatments. FIG. 3G is a graph showing the ratio of CD8⁺ IFN-γ⁺ T cells to Foxp3⁺ T cells in tumors that received the indicated treatments. FIG. 3H is a graph showing the results of the IFN-γ ELISPOT assay for IFN-γ-secreting MC38 cell antigen-specific CD8⁺ T cells from tumor draining lymph nodes according to the indicated treatments. FIG. 3I is a graph showing the qPCR analysis of mRNA levels of CXCL9, CXCL10 and CXCL11 in sorted CD11b⁺CD11c⁺ cells from tumors that received the indicated treatments. FIG. 3J is a graph showing the CD4⁺% in tumors that received the indicated treatment when iNOS activity was inhibited by an inhibitor (1400 W). FIG. 3K is a graph showing the CD8⁺% in tumors that received the indicated treatments when iNOS activity was inhibited by using an inhibitor (1400 W). FIG. 3L is a graph showing the % s of CD4⁺, CD8⁺, IFN-γ⁺CD4⁺ and IFN-γ⁺CD8⁺ T cells in tumors grown in a CCR2^{-/-} host which received a wild type (WT) or iNOS^{-/-} myeloid cell transfer. Cells were transferred one day prior to IR and tumors were harvested 8 days after control (oil) or combination treatment of IR and RA. *, p<0.05; **, p<0.01; ***, p<0.001, ns, not significant. Experiments were conducted 3 times with 3-mice per group. Data in FIGS. 3H and 3L are presented as mean±SEM and the rest of the panels are mean±SD. Results from representative experiments are shown.

FIGS. 4A-4H: T cells are important for antitumor efficacy of ionizing radiation (IR) and all-trans retinoic acid (RA) combination treatment and inflammatory macrophage (Inf-MAC) induction. FIG. 4A is a graph showing tumor growth curves (tumor volume in cubic millimeters (mm³) versus days after tumor inoculation) of murine colon adenocarcinoma (MC38) tumors established in wild type (WT) mice or Rag^{-/-} mice treated by oil, RA, 15 gray (Gy) IR+oil or 15Gy IR and RA combined. FIGS. 4B-4D are graphs showing MC38 tumor growth curves (tumor volume (mm³) versus days after tumor inoculation) when T cells were depleted during the indicated treatments of WT mice injected with (FIG. 4B) both anti-CD4 and anti-CD8 antibodies (CD4/CD6 depletion); (FIG. 4C) anti-CD8 antibodies (CD8 depletion); and (FIG. 4D) anti-CD4 antibodies (CD4 depletion). In CD4 depletion, 800 micrograms per dose per day (ug/dose/day) of RA was used. FIG. 4E is a graph showing the MC38 tumor growth curve tumor volume (mm³) versus days after tumor inoculation) when FTY720 was administered for 7 days from starting of the indicated treatment. FIG. 4F is a graph of inducible nitric oxide synthase (iNOS) expression (as a %) in myeloid cells of tumors in mice that received the indicated treatment when CD4, CD8 or CD4⁺CD8 were depleted. FIG. 4G is a graph showing the Inf-MAC level (as a ratio of total cells) in tumors treated with oil, IR and RA, or IR and RA and FTY720. FIG. 4H is a graph showing the tumor growth curves (tumor volume (mm³) versus days after tumor inoculation) of IR/RA during NK1.1 depletion. Anti-NK1.1 antibody was administered every 3 days for 3 doses from the start of the treatments. *, p<0.05; **, p<0.01; ***, p<0.001; ****, p<0.0001; ns, not significant. Experiments were repeated 3 times with 3-5 mice per group. Data in FIG. 4F are presented as mean±SD, the rest are presented as mean±SEM. Results from representative experiments are shown.

FIGS. 5A-5H: T cells promote the generation of inflammatory macrophages (Inf-MACs) through interferon gamma (IFN-γ) signaling in ionizing radiation (IR) and all-trans retinoic acid (RA) combination treatment. FIGS. 5A and 5B are graphs showing inducible nitric oxide synthase (iNOS) induction in bone marrow-derived dendritic cells (BMDC) in the presence of RA and/or IFN-γ. FIG. 5C is a graph showing IFN-γ levels (as picograms per gram tumor (pg/g tumor)) in tumors two days after receiving radiation. FIGS. 5D-5E are graphs showing the iNOS induction in myeloid cells of tumors treated with indicated treatments in the presence of anti-IFN-γ neutralizing antibody. FIG. 5F is a graph showing tumor necrosis factor factor-alpha (TNF-α) induction (as a percentage (%)) in myeloid cells of tumors in the presence of IFN-γ neutralizing antibody. FIG. 5G is a graph showing the murine colon adenocarcinoma (MC38) tumor growth curves (tumor volume in cubic millimeters (mm³) versus days after tumor inoculation) when the indicated treatments with or without IFN-γ neutralization were received. FIG. 5H is a graph showing iNOS-producing myeloid cell percentage (%) in tumors 4 days after treatment with vehicle or IR plus RA in Rag^{-/-} mice that received an adoptive transfer of WT or 1FN-γ^{-/-} T cells. *, p<0.05; **, p<0.01; ***, p<0.001; ****, p<0.0001; ns, not significant. Experiments were repeated 3 times with 3-5 mice per group. Data in FIGS. 5C, 5G, and are presented as mean±SEM, the rest are presented as mean±SD. Results from representative experiments are shown.

FIGS. 6A-6I: Programmed death ligand 1 (PD-L1) blockade enhances abscopal effects of all-trans retinoic acid (RA) and ionizing radiation (IR) combination treatment. FIG. 6A is a graph showing the growth curves of non-irradiated murine colon adenocarcinoma (MC38) tumors in two tumor model with primary tumors treated with oil, RA, 15 gray (Gy) IR+oil and 15Gy IR plus RA. FIGS. 6B and 6C are graphs showing inflammatory macrophage (Inf-MAC) production in non-irradiated tumors 8 days post IR. FIG. 6C shows Inf-MAC production quantified as a percentage (%) of total cells. FIGS. 6D and 6E are graphs showing the % changes in (FIG. 6D) CD4 and (FIG. 6E) CD8 levels during treatments in non-irradiated tumors. FIG. 6F is a graph showing interferon gamma (IFN-γ) producing spots of tumor antigen-specific CD8 T cells isolated from draining lymph nodes (DLN) of non-irradiated tumor. FIGS. 6G and 6H are graphs showing PD-L1 induction (as mean fluorescent intensity (MFI)) in (FIG. 6G) dendritic cells (DCs) and (FIG. 6H) tumor cells in non-irradiated tumors 3 days post starting of indicated treatments. FIG. 6I is a graph showing the growth curves (tumor volume in cubic millimeters (mm³) versus days after tumor inoculation) of non-irradiated tumors in MC38 2-tumor model when primary tumors received indicated treatment (oil, anti-PD-L1 antibodies, IR+RA, or IR+RA+anti-PD-L1-antibodies). *, p<0.05; **, p<0.01; ***, p<0.001; ns, not significant. Experiments were repeated 3 times with 3-5 mice per group. Data in FIG. 6I are presented as mean±SEM, and the rest are presented as mean±SD. Results from representative experiments are shown.

FIGS. 7A-7D: High inducible nitric oxide synthase (iNOS) expression in tumor tissue correlate to favorable survival and T cell signature in colon and kidney cancer patients. FIGS. 7A and 7B are graphs showing overall survival analysis (as percent survival) of kidney renal clear cell carcinoma (KIRC) (FIG. 7A) or colon adenocarcinoma (COAD) (FIG. 7B) cancer patients with either high or low messenger RNA (mRNA) expression for NOS2 (iNOS). Patients from each dataset were split into two groups by median expression level of NOS2. Normalized Gene expression (RNAseq) and corresponding clinical data of patients were obtained from The Cancer Genome Atlas (TCGA). Survival curves were compared by log rank (Mantel-COX) test. FIG. 7C is a series of graphs showing the correlation between expression of NOS2 and CD3E, CD8a, IFN-γ and PRF1 in colorectal adenocarcinoma patients (n=592) from TCGA PanCancer data. Spearman r and P values are included in the figures. Gene expression levels are presented in the log 2 form. FIG. 7D is a schematic diagram summarizing a proposed mechanism that in combination treatment of radiation (IR) and all-trans retinoic acid (RA), the infiltrating monocytes, which are induced by radiation, are transformed into iNOS/tumor necrosis factor-alpha (TNF-α) producing inflammatory macrophages (Inf-MACs). Through iNOS production, the Inf-MACs enhance enhance CD4+ and CD8⁺ T cell infiltration and the newly arrived T cells produce more IFN-γ to induce an even higher level of Inf-MACs. The positive feedback loop between Inf-MACs and T cells amplified antitumor innate and adaptive immunity and leads to superior tumor control than any single treatment alone.

FIGS. 8A-8B: In vivo anti-cancer efficacy of all-trans retinoic acid (ATRA)-sensitized radiotherapy on a murine colon adenocarcinoma (MC38) tumor model. Treatment began on day 7 after tumor inoculation when the tumor reached a volume of 100-120 cubic millimeters (mm³). Mice were administrated with oral gavage of oil or ATRA followed by X-ray irradiation (n=5). X-ray irradiation was carried out on mice after the oral gavage for 3 consecutive days at a dose of 2 gray (Gy)/fraction. FIG. 8A is a graph showing tumor growth curves (tumor volume in mm³ versus day post treatment) of MC38 tumor-bearing C57BL/6 mice after different treatments (oil without irradiation (Oil(−)), oil with irradiation (Oil (+)), or ATRA with irradiation (ATRA (+)). FIG. 8B is a graph showing body weight change percentage (%) of MC38 tumor-bearing C57BL/6 mice after different treatments (oil without irradiation (Oil(−)), oil with irradiation (Oil (+)), or ATRA with irradiation (ATRA (+)). n=5.

FIGS. 9A-9D: In vivo anti-cancer efficacy of all-trans retinoic acid (ATRA)-sensitized radiotherapy on a murine colorectal carcinoma (CT26) tumor model. Treatment began on day 7 after tumor inoculation when the tumor reached a volume of 100-120 cubic millimeters (mm³). Mice were administrated with oral gavage of oil or ATRA with (+) or without (−) X-ray irradiation (n=5). X-ray irradiation was carried out on mice after the oral gavage for 6 consecutive days at a dose of 1 gray (Gy)/fraction. FIG. 9A is a graph showing tumor growth curves (tumor volume in mm³ versus day post treatment) of CT26 tumor-bearing BALB/c mice after treatments (oil without irradiation (Oil(−)), oil with irradiation (Oil (+)), or ATRA with irradiation (ATRA (+)). FIG. 9B is a graph showing body weight change percentage (%) of CT26 tumor-bearing BALB/c mice after the same treatments as described for FIG. 9A. FIG. 9C is a drawing of excised tumors corresponding to the treatments described for FIG. 9A at the endpoint. FIG. 9D is a graph showing tumor weights at the endpoint for the tumors shown in FIG. 9C. n=5.

FIGS. 10A-10B: In vivo anti-cancer efficacy of all-trans retinoic acid (ATRA)-sensitized radiotherapy on Rag1^{-/-} mice models. Treatment began on day 7 after tumor inoculation when the tumor reached a volume of 100-120 cubic millimeters (mm³). Mice were administrated with oral gavage of oil or ATRA with (+) or without (−) X-ray irradiation (n=5). X-ray irradiation was carried out on mice after oral gavage for 3 consecutive days at a dose of 2 gray (Gy)/fraction. FIG. 10A is a graph showing tumor growth curves (tumor volume in mm³ versus day post treatment) of murine colorectal carcinoma (MC38) tumor-bearing Rag1^{-/-} mice after treatment with oil or ATRA and with or without irradiation. n=5. FIG. 10B is a graph of body weight change percentage (%) of MC38 tumor-bearing Rag1^{-/-} mice after treatment with oil or ATRA and with or without irradiation. n=5.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. Although any methods, devices, and materials similar or equivalent to those described herein can be used in the practice or testing of the presently disclosed subject matter, representative methods, devices, and materials are now described. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist, unless otherwise indicated.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a chemotherapeutic agent” includes a plurality of such chemotherapeutic agents, and so forth.

Unless otherwise indicated, all numbers expressing quantities of size, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), dose, weight, concentration, or percentage is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” can refer to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C1-8 alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C1-8 straight-chain alkyls. In other embodiments, “alkyl” refers, in particular, to C1-8 branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. In some embodiments, there can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀; —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “aralkyl” as used herein refers to a -alkyl-aryl group, which can be substituted or unsubstituted. An exemplary aralkyl group is benzyl.

The terms “carboxylic acid” and “carboxylate” refer to the groups —C(═O)OH and —C(═O)—O⁻.

The term “ester” refers to a derivative of a carboxylic acid, wherein the hydrogen atom of the OH group is replaced by a substituted or unsubstituted alkyl, aralkyl, or aryl group.

The term “amide” refers to a derivative of a carboxylic acid wherein the OH group is replaced by an amino group. The term “amino” refers to the group —N(R)₂ wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. “Arylamine” and “aminoaryl” refer to the group —N(R)₂ wherein each R is H, aryl, or substituted aryl, and wherein at least one R is aryl or substituted aryl, e.g., aniline (i.e., —NHC₆H₅).

As used herein, a “derivative” of a compound refers to a chemical compound that can be produced from another compound of similar structure in one or more steps, as in replacement of H by an alkyl, acyl, or amino group.

The term “retinoid” as used herein refers to naturally occurring, semi-synthetic, and synthetic compounds belonging to a class of vitamin A derivatives and analogs. For example, retinoids are vitamin A derivatives that exhibit biological activity against vitamin A deficiency and/or bind one or more retinoid receptors. In some embodiments, the retinoid is a vitamin A derivative consisting of four isoprenoid units joined in a head-to-tail manner. Examples of retinoids include, but are not limited to, a retinoic acid (e.g. all-trans retinoic acid (ATRA or RA, also known as tretinoin), 9-cis-retinoic acid (also known as alitretinoin), 13-cis-retinoic acid (also known as isotretinoin), 9,13-di-cis-retinoic acid), esters and amides of retinoic acids (e.g., fenretinide), retinal, retinol, 4-hydroxy-retinoic acid, 4-oxo-retinoic acid, 18-hydroxy-retinoic acid, 5,6-epoxy-retinoic acid, etretinate, bexarotene, lazarotene, trifarotene, benzoic acid-terminated retinoids and their heterocyclic analogs such as TTNPB, TTAB, AM80, AM580, SRI 1251, SRI 1247, CD666, CD367, chalcone-4-carboxylic acids, flavone-4′-carboxylic acids, etc. (Loeliger et al., 1980, Eur. J. Med. Chem-Dhim. Ther. 15:9), (Kagechika et al, 1989, J. Med. Chem. 32:834), (Dawson, et al. 1995, J. Med. Chem. 38:3368) as well as napthalenecarboxylic acid-terminated retinoids such as TTNN, CD437, CD417 or adapalene (Dawson et al., 1983, J. Med. Chem. 26:1653), (Dhar et al., 1999, J. Med. Chem. 42:3602) and many other carboxylic acid retinoids (AGN 190299 or tazarotenic acid and RQ 10-9359 or acitretin). These and other retinoids are described, for example, in U.S. Pat. No. 11,077,139, the disclosure of which is incorporated herein by reference in its entirety. The term retinoids can also refer to biologically active metabolites of the above-described compounds.

The term “cancer” as used herein refers to diseases caused by uncontrolled cell division and/or the ability of cells to metastasize, or to establish new growth in additional sites. The terms “malignant”, “malignancy”, “neoplasm”, “tumor,” “cancer” and variations thereof refer to cancerous cells or groups of cancerous cells.

Particular types of cancer include, but are not limited to, skin cancers (e.g., melanoma), connective tissue cancers (e.g., sarcomas), adipose cancers, breast cancers, head and neck cancers, lung cancers (e.g., mesothelioma), stomach cancers, pancreatic cancers, ovarian cancers, cervical cancers, uterine cancers, anogenital cancers (e.g., testicular cancer), kidney cancers, bladder cancers, colon cancers, prostate cancers, central nervous system (CNS) cancers, retinal cancer, blood, neuroblastomas, multiple myeloma, and lymphoid cancers (e.g., Hodgkin's and non-Hodgkin's lymphomas).

The term “metastatic cancer” refers to cancer that has spread from its initial site (i.e., the primary site) in a patient's body.

The terms “anticancer drug”, “chemotherapeutic”, and “anticancer prodrug” refer to drugs (i.e., chemical compounds) or prodrugs known to, or suspected of being able to treat a cancer (i.e., to kill cancer cells, prohibit proliferation of cancer cells, or treat a symptom related to cancer). In some embodiments, the term “additional chemotherapeutic” as used herein refers to a non-retinoid that is used to treat cancer and/or that has cytotoxic ability. Some traditional or conventional chemotherapeutic agents that can be used as “additional chemotherapeutic” agents can be described by mechanism of action or by chemical compound class, and can include, but are not limited to, alkylating agents (e.g., melphalan), anthracyclines (e.g., doxorubicin), cytoskeletal disruptors (e.g., paclitaxel), epothilones, histone deacetylase inhibitors (e.g., vorinostat), inhibitors of topoisomerase I or II (e.g., irinotecan or etoposide), kinase inhibitors (e.g., bortezomib), nucleotide analogs or precursors thereof (e.g., methotrexate), peptide antibiotics (e.g., bleomycin), platinum based agents (e.g., cisplatin or oxaliplatin), and vinka alkaloids (e.g., vinblastine).

The term “solid tumor” as used herein refers to a tumor comprising a solid mass of cancer cells. Solid tumors include sarcomas, carcinomas and some lymphomas. Solid tumors can occur in different organs, e.g., lung, breast, prostate, colon, rectum, and bladder. In contrast, “liquid tumors” typically occur in the blood, bone marrow, and lymph nodes and include leukemias, myelomas, and some lymphomas.

The term “abscopal” refers to a therapeutic effect on a tumor in a part of a subject's or patient's body that is not directly targeted by local therapy (e.g., local RT). In some embodiments, the therapeutic effect is shrinkage or disappearance of the non-locally targeted tumor.

The term “fractionated” as used herein in reference to RT refers to a total radiation dose for treating a tumor being split into a plurality (e.g., 2, 3, 4, 5, 6, 7, 8, 9, 10, 14, 21, 28, etc.) of small doses or fractions that are administered at different times (e.g., on different days, typically over the course of one or more weeks), e.g., to help allow normal (non-diseased) cells to repair between fractions and reduce the side effects that would have occurred if the total radiation dose were administered at one time.

### II. General Considerations

Radiotherapy (RT) is employed in 50%-60% of patients with cancer. However, local and distal failure in patients with large localized tumors often occurs. The efficacy of IR depends in part on direct cytotoxic effects of DNA damage on the tumor and stroma, and in part on innate and adaptive immune responses (1-4). Radiation can influence antitumor immunity through multiple mechanisms. For example, radiation-induced cellular stress and death can generate damage-associated molecular pattern (DAMP) molecular signals, DNA, chemokines, and cytokines, which provide key links between innate and adaptive immunity (4-6). Emerging evidence also demonstrates that radiation increases the expression of immune suppressive molecules such as IL-10, PD-L1, and CTLA-4, in addition to elevating regulatory T cells (T_{reg}) and myeloid-derived suppressor cell (MDSC) numbers within the tumor microenvironment (7-10). These observations have led to many clinical trials employing IO (Immuno-Oncology) agents in combination with radiotherapy. However, the optimization of IR and IO interactions has yet to be defined (11, 12). Therefore, exploring new strategies to combine radiotherapy with immunotherapy can provide an opportunity to improve the clinical outcomes for patients with cancer (4, 13).

All-trans retinoic acid (ATRA) is an active metabolite of vitamin A that is required for a wide range of physiological processes and plays a significant role in many pathologies (14-16). ATRA use in the treatment of acute promyelocytic leukemia (APL) is considered to be a paradigm shift, in which it works by targeting the oncoproteins PML-RARα and Pin1 to promote differentiation of malignant myeloid cells (17). However, there is a limited body of evidence on the clinical utility of ATRA to treat other leukemias and solid tumors (18).

The effect of combined IR and ATRA for the treatment of solid tumors has not been widely explored. Despite significant efforts over a century, the Food and Drug Administration (FDA) has not approved any non-toxic compound as a radioenhancer. According to one aspect of the presently disclosed subject matter, it is demonstrated that combining local ablative IR with ATRA significantly enhances the effect of the IR and potentiates checkpoint blockade immunotherapy. As described in the examples, the combination of IR and ATRA significantly inhibits the growth of local and distal (non-IR-targeted) tumors. As further described herein, combination treatment also leads to a dramatic increase of inducible nitric oxide synthase (iNOS)/TNF-α-producing myeloid cells in both local and distal tumors. These myeloid cells, whose generation involves IFN-γ and adaptive immunity, also in turn promote T cell infiltration through a positive feedback loop. Additionally, the combination of IR and ATRA enhances fractionated, low-dose RT to eradicate tumors in subjects.

Accordingly, in some embodiments, the presently disclosed subject matter relates to the use of a retinoid in combination with X-ray irradiation and/or other therapeutic agents, e.g., immune checkpoint inhibitors, for treating disease (e.g., cancer). In some embodiments, the combination can provide synergistic anticancer therapeutic efficacy.

Thus, in some embodiments, the presently disclosed subject matter provides a method of treating a cancer in a subject in need thereof. In some embodiments, the method comprises: administering to the subject a retinoid or a pharmaceutically acceptable salt thereof; and exposing at least a portion of the subject to ionizing irradiation energy, such as X-rays and/or protons.

The retinoid can be a single retinoid or a mixture of two or more retinoids, such as those described hereinabove. In some embodiments, the retinoid is selected from the group including, but not limited to, retinol, retinal, a retinoic acid, an ester or amide of a retinoic acid, a metabolite of a retinoic acid, or mixtures thereof. In some embodiments, the retinoid is selected from ATRA, 9-cis-retinoic acid, 13-cis retinoic acid, fenretinide, retinal, 4-hydroxy-retinoic acid, 4-oxo-retinoic acid, 18-hydroxy-retinoic acid, 5,6-epoxy-retinoic acid, and mixtures thereof. In some embodiments, the retinoid comprises or consists of a retinoic acid or a pharmaceutically acceptable salt thereof. In some embodiments, the retinoid comprises or consists of ATRA or a pharmaceutically acceptable salt thereof.

In some embodiments, the cancer is not acute promyelocytic leukemia (APL). In some embodiments, the cancer is not a leukemia. In some embodiments, the cancer is a solid tumor cancer. In some embodiments, the cancer is selected from the group including, but not limited to, a skin cancer, a connective tissue cancer, an adipose cancer, a breast cancer, a head and neck cancer, a lung cancer, a stomach cancer, a pancreatic cancer, an ovarian cancer, a cervical cancer, a uterine cancer, an anogenital cancer, a kidney cancer, a bladder cancer, a colon cancer, a prostate cancer, a central nervous system (CNS) cancer, a retinal cancer, a neuroblastoma, and a lymphoid cancer. In some embodiments, the cancer is a lung, breast, prostate, kidney, colon, or bladder cancer, including a solid tumor cancer of one of these cancers. In some embodiments, the cancer is a colon cancer or a kidney cancer, including a solid tumor cancer of one of these cancers.

In some embodiments, the subject in need of treatment is a mammal. In some embodiments, the subject is a human.

In some embodiments, the method further comprises administering to the subject an additional therapeutic agent or treatment. In some embodiments, the additional therapeutic agent or treatment is selected from an immunotherapy agent and/or a cancer treatment. In some embodiments, the cancer treatment is selected from the group comprising surgery, chemotherapy (e.g., administration of an additional chemotherapeutic compound in addition to the retinoid), toxin therapy, cryotherapy and gene therapy. For example, the additional chemotherapeutic compound can be selected from the group including, but not limited to, a platinum complex, such as cisplatin, oxaliplatin, carboplatin, or a prodrug thereof; doxorubicin; daunorubicin; docetaxel; mitoxanthrone; paclitaxel; digitoxin; gem citabine; methotrexate; leucovorin; pemetresed disodium; vinblastine; vincristine; vindesine; cytarabine; azathioprine; melphalan; imitnib; anastrozole; letrozole; etoposide; vinorelbine; digoxin, and septacidin. In some embodiments, more than one additional chemotherapeutic agent can be used.

In some embodiments, the additional therapeutic agent or treatment comprises an immunotherapy agent. In some embodiments, the immunotherapy agent is selected from the group including, but not limited to, an anti-CD52 antibody, an anti-CD20 antibody, an anti-CD20 antibody, anti-CD47 antibody an anti-GD2 antibody, a radiolabeled antibody, an antibody-drug conjugate, polysaccharide K, a neoantigen, an anti-LAG3 antibody, an anti-4-IBB antibody, an anti-TIM3 antibody and a cytokine. In some embodiments, the cytokine is an interferon, an interleukin, or tumor necrosis factor alpha (TNF-α). In some embodiments, the cytokine is selected from the group comprising IFN-α, INF-γ, IL-2, IL-12 and TNF-α. In some embodiments, the immunotherapy agent is selected from the group comprising Alemtuzumab, Ofatumumab, Rituximab, Zevalin, Adcetris, Kadcyla and Ontak. In some embodiments, the immunotherapy agent is selected from the group comprising a PD-1 inhibitor or a PD-L1 inhibitor, such as, but not limited to, BMS-936559 or BMS-936558 from Bristol-Myers Squibb, MPDL3280A from Genentech, MK-3475 from Merck, CT-011 from Curetech, and MEDI4736 from Medlmmune; a CTLA-4 inhibitor (e.g., ipilimumab, tremelimumab); an indoleamine-2,3-dioxygenase (IDO) inhibitor; and a CCR7 inhibitor. The IDO inhibitor can be any suitable IDO inhibitor, such as, but not limited to oxadiazole and other heterocyclic IDO inhibitors, e.g., as reported in U.S. Patent Application Publication Nos. 2006/0258719 and 2007/0185165, which are incorporated herein by reference in their entireties. IDO inhibitors also include those described in PCT Publication WO 99/29310, incorporated herein by reference in its entirety, which reports methods for altering T cell-mediated immunity comprising altering local extracellular concentrations of tryptophan and tryptophan metabolites, BMS-986205 from Bristol-Myers Squibb or F001287 from Flexus, epacadostat from Incyte Corp., indoximod, 10-102, EOS-200271, HTI-1090, 10-101, KHK-2455, 1-methyl-DL-tryptophan, p-(3-benzofuranyl)-DL-alanine, p-[3-benzo(b)thienyl]-DL-alanine, and 6-nitro-L-tryptophan), and IDO inhibitors described in WO 03/087347, incorporated herein by reference in its entirety, also published as European Patent 1501918, which describes methods of making antigen-presenting cells for enhancing or reducing T cell tolerance. Compounds having IDO inhibitory activity are further reported in WO 2004/094409, and in U.S. Patent Application Publication No. 2004/0234623, each of which is incorporated herein by reference in its entirety, and each of which is directed to methods of treating a subject with a cancer or an infection by the administration of an inhibitor of indoleamine-2,3-dioxygenase in combination with other therapeutic modalities. In some embodiments, the small molecule inhibitors are those disclosed in U.S. Pat. No. 8,088,803, which is incorporated by reference in its entirety herein. In some embodiments, the immunotherapy agent is an immune checkpoint inhibitor (e.g., an antibody immune checkpoint inhibitor, such as, but not limited to, a PD-1/PD-L1 antibody, a CTLA-4 antibody, an OX40 antibody, a TIM3 antibody, a LAG3 antibody, an anti-CD47 antibody). In some embodiments, the immune checkpoint inhibitor is selected from the group consisting of a PD-1 inhibitor, a PD-L1 inhibitor, a CTLA-4 inhibitor, an IDO inhibitor, a CCR7 inhibitor, an OX40 inhibitor, a TIM3 inhibitor, and a LAG3 inhibitor, optionally wherein the immune checkpoint inhibitor is a PD-L1 inhibitor.

The retinoid can be administered via any suitable route, e.g., via injection directly to a tumor site, orally, intraperitoneally, intravenously, subcutaneously, etc. In some embodiments, the retinoid is administered orally.

In some embodiments, the patients are irradiated with a linear accelerator (LINAC), using conventional techniques, Intensity-Modulated Radiation Therapy (IMRT), Image Guided Radiation Therapy (IGRT), or Stereotactic Body Radio Therapy (SBRT), a ⁶⁰Co radiation source, an implanted radioactive seed such as the ones used in brachytherapy, an orthovoltage or supervoltage X-ray irradiator, a high energy electron beam generated from LINAC, or a proton source.

In some embodiments, the γ-rays generated by a LINAC pass through an energy modulator (filter) before irradiating the patient, optionally wherein the filter comprises one or more element(s) with atomic number(s) of at least 20, further optionally wherein the filter comprises copper. In some embodiments, the filter has a thickness that is less than about 5 mm, less than about 4 mm, less than about 3 mm, less than about 2 mm, less than about 1 mm, less than about 0.5 mm, less than about 0.4 mm, less than about 0.3 mm, less than about 0.2 mm, or less than about 0.1 mm.

In some embodiments, X-rays are generated by placing radioactive sources inside the patient on a temporary or permanent basis. In some embodiments, a nanoparticle composition is injected along with the implantation of a radioactive source.

The radiation dosage regimen is generally defined in terms of gray (Gy) or sieverts, time and fractionation and can be carefully defined by the radiation oncologist. The amount of radiation a subject receives can depend on various considerations, such as the location of the tumor in relation to other critical structures or organs of the body, and the extent to which the tumor has spread. One illustrative course of treatment for a subject undergoing radiation therapy is a treatment schedule with a total dose of about 5 Gy, about 10 Gy, about 15 Gy, about 20 Gy, about 25 Gy, about 30 Gy, about 35 Gy, about 40 Gy, about 45 Gy, about 50 Gy, about 55 Gy, about 60 Gy, about 65 Gy, about 70 Gy, about 75 Gy, or about 80 Gy or any derivable range therein. The radiation dose can be administered to the subject in a single daily fraction of about 1.0 Gy to about 5.0 Gy for 1 day, 2 days, 3 days, 4 days, or 5 days a week for one to two weeks or about 5.0 Gy to about 10.0 Gy for 1 day, 2 days, 3 days, 4 days, or 5 days a week for one week. One Gy refers to 100 rad of dose. Illustrative dosages used for photon-based radiation are measured in Gy, and in an otherwise healthy subject (that is, little or no other disease states present such as high blood pressure, infection, diabetes, etc.) for a solid epithelial tumor ranges from about 60 Gy to about 80 Gy, and for a lymphoma ranges from about 20 Gy to about 40 Gy. Illustrative preventative (adjuvant) doses are typically given at about 45 Gy to about 60 Gy in about 1.8 Gy to about 2 Gy fractions for breast, head, and neck cancers.

The method can comprise administering the retinoid to the subject at about the same time as, or later than the administration of the radiation. In some embodiments, the retinoid can be administered to the subject prior to a dose or course of radiation.

As noted hereinabove, the method can further comprise administering to the subject an additional therapeutic agent or treatment. In some embodiments, the additional therapeutic agent or treatment is an immunotherapy agent or an additional cancer treatment selected from surgery, chemotherapy (e.g., a chemotherapeutic agent other than and in addition to the retinoid), toxin therapy, cryotherapy, and gene therapy. In some embodiments, the additional chemotherapeutic agent can be co-administered with the retinoid, e.g., in the same formulation or in different formulations administered at about the same time. In some embodiments, the additional therapeutic agent is an immunotherapeutic agent. In some embodiments, the additional therapeutic agent is an immune checkpoint inhibitor, such as, but not limited to, a PD-1 inhibitor, a PD-L1 inhibitor, a CTLA-4 inhibitor, an IDO inhibitor, a CCR7 inhibitor, an OX40 inhibitor, a TIM3 inhibitor, and a LAG3 inhibitor. The immunotherapy agent or other treatment can be administered prior to, at the same time as, or later than the administration of the retinoid and/or irradiation. For example, in some embodiments, administration of a retinoid and X-ray irradiation treatment can be performed prior to surgery to reduce the size of a tumor.

In some embodiments, the exposing of at least a portion of the subject to ionizing irradiation is performed by exposing said at least a portion of the subject to a fraction of a total dose of ionizing irradiation energy on two or more separate days until said at least a portion of the subject is exposed to said total dose of ionizing irradiation energy. In some embodiments, the two or more separate days are two or more consecutive days or two or more consecutive weekdays (i.e., Monday, Tuesday, Wednesday, Thursday, and Friday). In some embodiments, the two or more separate days are spread over the course of one, two, three, four, or more weeks.

In some embodiments, the retinoid and IR combination of the presently disclosed subject matter provides enhanced tumor growth control compared to a treatment comprising the administering (i.e., the retinoid administration) alone or the exposing (i.e., the IR) alone. In some embodiments, the administration of the retinoid alone does not provide any significant or detectable anti-tumor effect. For example, in some embodiments, the administration of the retinoid comprises administering a retinoid to the subject at a dose that, if administered absent the exposing step, would not result in any significant or detectable anti-tumor effect. In some embodiments, an ARTA dose of about mg/m² to about 150 mg/m² daily (e.g., about 45 mg/m², about 60 mg/m², about 75 mg/m², about 90 mg/m², about 105 mg/m², about 120 mg/m², about 135 mg/m², or about 150 mg/m² daily) can be used in combination with radiotherapy.

In some embodiments, the retinoid and IR combination of the presently disclosed subject matter provides an abscopal effect, i.e., provides enhanced tumor growth control of a tumor distal to a tumor directly targeted by the IR and/or retinoid administration. In some embodiments, the method provides partial or total eradication of the distal tumor via eliciting systemic antitumor immunity.

In some embodiments, the combination of IR and retinoid provides comparable or enhanced (e.g., greater) tumor growth control (e.g., tumor shrinkage) using a lower total dose of ionizing radiation energy compared to a treatment involving exposing the subject to IR alone or a treatment involving IR, but not administration of retinoid. In some embodiments, the presently disclosed subject matter provides for comparable or enhanced tumor growth control to IR alone using a relatively small fractionated dose (i.e., a relatively small fractionated dose for humans), such as about 160 centigray (cGy)/day to about 600 cGy/day (e.g., about 160 cGy/day, about 200 cGy/day, about 240 cGy/day, about 280 cGy/day, about 320 cGy/day, about 360 cGy/day, about 400 cGy/day, about 440 cGy/day, about 480 cGy/day, about 520 cGy/day, about 560 cGy/day, or about 600 cGy/day).

In some embodiments, the presently disclosed combination of retinoid and IR provides an increase in iNOS-producing myeloid cells in the subject. For example, in some embodiments, the combination provides an increased level of CD11b+iNOS+ cells in a tumor in the subject. In some embodiments, the combination of retinoid and IR provides an increase in TNF-α-producing myeloid cells in the subject. In some embodiments, the combination of retinoid and IR provides protection from tumor recurrence (e.g., compared to a treatment with IR alone) via antitumor immune memory.

### III. Pharmaceutical Compositions

In some embodiments, the presently disclosed subject matter provides a composition comprising a retinoid (e.g., ATRA) as described herein and a pharmaceutically acceptable carrier, e.g., a pharmaceutically acceptable carrier that is pharmaceutically acceptable in humans, e.g., for use in the presently disclosed method. In some embodiments, the composition can also include other components, such as, but not limited to anti-oxidants, buffers, bacteriostatics, bactericidal antibiotics, suspending agents, thickening agents, and solutes that render the composition isotonic with the bodily fluids of a subject to whom the composition is to be administered.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the subject; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in one example in the range of about 0.1 to about 10 mg/ml, in another example about 2.0 mg/ml; and/or mannitol or another sugar, for example in the range of about 10 to about 100 mg/ml, in another example about 30 mg/ml; and/or phosphate-buffered saline (PBS).

It should be understood that in addition to the ingredients particularly mentioned above, the formulations of the presently disclosed subject matter can include other agents conventional in the art having regard to the type of formulation in question. For example, sterile pyrogen-free aqueous and non-aqueous solutions can be used.

As noted above, in some embodiments, the retinoid can be provided as a pharmaceutically acceptable salt. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, and magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

The methods and compositions disclosed herein can be used on a sample either in vitro (for example, on isolated cells or tissues) or in vivo in a subject (i.e. living organism, such as a patient). In some embodiments, the subject is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (e.g., mice, rats, hamsters, gerbils, etc.) and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

Suitable methods for administration of a composition of the presently disclosed subject matter include, but are not limited to intravenous and intratumoral injection, oral administration, subcutaneous administration, intraperitoneal injection, intracranial injection, and rectal administration. Alternatively, a composition can be deposited at a site in need of treatment in any other manner, for example by spraying a composition within the pulmonary pathways. The particular mode of administering a composition of the presently disclosed subject matter depends on various factors, including the distribution and abundance of cells to be treated and mechanisms for metabolism or removal of the composition from its site of administration. For example, relatively superficial tumors can be injected intratumorally. By contrast, internal tumors can be treated following intravenous injection.

In one embodiment, the method of administration encompasses features for regionalized delivery or accumulation at the site to be treated. In some embodiments, a composition is delivered intratumorally. In some embodiments, selective delivery of a composition to a target is accomplished by intravenous injection of the composition followed by irradiation (e.g., X-ray or proton irradiation) of the target.

For delivery of compositions to pulmonary pathways, compositions of the presently disclosed subject matter can be formulated as an aerosol or coarse spray. Methods for preparation and administration of aerosol or spray formulations can be found, for example, in U.S. Pat. Nos. 5,858,784; 6,013,638; 6,022,737; and 6,136,295.

In some embodiments, an effective dose of a composition of the presently disclosed subject matter is administered to a subject. An “effective amount” is an amount of the composition sufficient to produce detectable treatment (e.g., tumor size reduction, pain relief, reduction of the concentration of a disease-related blood marker, etc.). Actual dosage levels of constituents of the compositions of the presently disclosed subject matter can be varied so as to administer an amount of the composition that is effective to achieve the desired effect for a particular subject and/or target. The selected dosage level can depend upon the activity (e.g., the IC₅₀ of the therapeutic components in certain cell types (e.g., cancer cell lines) of the composition and the route of administration.

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art.

A dose may be administered on an as needed basis or about every 1 hour, 2 hours, 3 hours, 4 hours, 5 hours, 6 hours, 7 hours, 8 hours, 9 hours, 10 hours, 11 hours, 12 hours, 18 hours, or 24 hours (or any range derivable therein) or 1 time, 2 times, 3 times, 4 times, 5 times, 6 times, 7 times, 8 times, 9 time, or 10 times per day (or any range derivable therein). A dose can be first administered before or after symptoms are exhibited by the subject; after a clinician evaluates the subject for the disease; or before, at about the same time as, or after administration of a second treatment (chemotherapy, radiation therapy, immunotherapy). In some embodiments, the patient is administered a first dose of a retinoid compound, such as ATRA, about 1 hour, about 2 hours, about 3 hours, about 4 hours, about 5 hours, about 6 hours, about 7 hours, about 8 hours, about 9 hours, about 10 hours, about 11 hours, about 12 hours (or any range derivable therein) or about 1 day, about 2 days, about 3 days, about 4 days, or about 5 days after the subject exhibits signs or symptoms of the disease (or any range derivable therein). The subject can be treated for about 1 day, about 2 days, about 3 days, about 4 days, about 5 days, about 6 days, about 7 days, about 8 days, about 9 days, or about 10 or more days (or any range derivable therein) or until symptoms of an the disease, such as tumor size, have disappeared or been reduced or after about 6 hours, about 12 hours, about 18 hours, or about 24 hours or about 1 day, about 2 days, about 3 days, about 4 days, or about 5 days after symptoms of the disease have disappeared or been reduced.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Materials and Methods for Examples 2-7

The effect of combined IR and retinoid treatment and the relationship between Inf-MACs and IFN-γ⁺ T cells were assessed in MC38-tumor-bearing mice (WT, iNOS KO, IFN-γ KO, CCR2 KO, RAG KO), treated with anti-CD4, anti-CD8, anti-NK, anti-IFN-γ, anti-PD-L1 or anti-GM-SCF neutralizing/blocking antibodies or the FTY720 and 1400 W inhibitors. Sample sizes for flow analysis, protein and gene expression analysis (n=3 to 5, repeated two to five times), tumor monitoring (n=4 to 7, repeated three times), and the choices of time points for analysis were based on previous studies (10, 39, 47). Investigators were blinded when performing and analyzing the data. For tumor growth monitoring, age-matched mice were assigned to experimental cohorts based on randomized matching tumor volumes, and data presented include all outliers. Investigators were not blinded when monitoring tumor burden. Biological replicates are indicated in the figure legends by “n.” Three or more independent trials were performed.

Mice, Cell lines and Reagents

Six- to eight-week-old C57BL/6J mice were purchased from Envigo (Indianapolis, Indiana, United States of America). Rag1 knockout mice, iNOS knockout, IFN-γ knockout and CCR2 knockout mice were from The Jackson Laboratory (Bar Harbor, Maine, United States of America). Mice were maintained under pathogen-free conditions. MC38, B16/F1, Renca, and LLC cell lines were acquired from American Type Culture Collection (ATCC, Manassas, Virginia, United States of America), tested free of murine pathogen and authenticated. Cells were cultured in DMEM medium with 10% FCS and 1% of penicillin/streptomycin (Pen/Strep) antibiotics. RA (Cat #PHR1187) was purchased from Sigma-Aldrich (St. Louis, Missouri, United States of America). 1400 W (i.e., N-[[-(aminomethyl)phenyl]methyl]-ethanimidamide dihydrochloride) was purchased from Cayman Chemical Company (Ann Arbor, Michigan, United States of America) (CAS #214358-33-5) and prepared in PBS before injection. Depleting or blocking antibodies against PD-L1 (6E0101), CD8α (6E0004-1), CD4 (BP0003-1), NK1.1 (6E0036), GM-CSF (6E0259), CD11 b (6E0007) and IFN-γ (6E0055) were purchased from BioXCell (Lebanon, New Hampshire, United States of America). Anti-mouse Pacific Blue-anti-CD45 (103126), fluorescein isothiocyanate (FITC)-anti-CD45 (103108), PE-anti-CD4 (116005), APC/CY7-anti-CD8α (100714), APC-anti-IFN-γ (505810), Alexa Fluor 700-anti-CD44(103025), Brilliant Violet 605-antiCD62L (104437), Zombie UV (77474), Brilliant Violet 605-anti-CX3CR1(149027), Brilliant Violet 650-anti-XCR1 (148220), Brilliant Violet-510-anti-CD24(101831), Brilliant Violet 711-anti-CD64 (139311), Brilliant Violet 785-anti-F4/80 (123141), Pacific Blue-anti-CD11b (101224), PerCP-Cy5.5-anti-1-A/I-E (107626), PE-Cy7-anti-CD11c (117318), FITC-anti-Ly6C (128006), PE-Dazzle 594-anti-CD103 (121429), Alexa Fluor 700-anti-CD206 (141734), APC-Cy7-anti-SIRPα (144017), and Alexa Fluor 488-anti-Ki-67 were purchased from BioLegend (San Diego, California, United States of America). Alexa Fluor 532-anti-CD45 (58-0451-82), PerCP-eFluor710-anti-Flt3 (46-1351-82), PE-anti-iNOS (12-5920-82), and APC-anti-TNF (17-7321-81) are from ThermoFisher Scientific (Waltham, Massachusetts, United States of America). PE-anti-iNOS (D6B6S, 14792) and Griess Reagent Nitrite Measurement Kit (13547) were from Cell Signaling Technology (Danvers, Massachusetts, United States of America). CD11c isolation kit (18780), and CD8 T cell selection kit (18953) were purchased from STEMCELL Technologies (Vancouver, Canada). Nitric oxide detection kit (ADI-917-020), and FTY720 (i.e., fingolimod hydrochloride; BML-SL233-0025) were purchased from Enzo Life Sciences, Inc. (Farmingdale, New York, United States of America). A mouse chemokine assay sold under the tradename LEGENDPLEX™ (740451) and mouse inflammation panel (740446) were purchased from BioLegend (San Diego, California, United States of America). Mouse IFN-γ CBA Flex Set (BDB558296) and Mouse IFN-γ ELISPOT Set (551083) were from Fisher Scientific (Hampton, New Hampshire, United States of America).

### Tumor Growth and Treatments

1×10⁶ MC38, B16/F1, Renca or CT26 cells were subcutaneously implanted into flanks of mice. Tumors were irradiated using a Phillips IR250 orthovoltage X-ray generator (Phillips, Amsterdam, the Netherlands) operating at 250 kV 15 mA. Subcutaneous local tumors were irradiated by shielding bodies with a cylindrical lead cover with only flank tumors exposed for radiation. ATRA (400 μg/mice/day) was administered by oral gavage for 10 days. For the two-tumor model, 1×10⁶ and 0.7×10⁶ MC38 cells were subcutaneously implanted into the right and left flank of mice respectively. For the tumor re-challenge assay, both the cured mice and the naive mice were re-challenged with 5×10⁶ MC38 or 1×10⁶ LLC cells respectively. Tumors were measured along two diameters (length and width) and depth twice weekly and volumes were calculated as length×width×depth/2. For CD8 and CD4 T cell depletion experiments, 200 μg antibodies were injected i.p. on day 0, 2, 5 and 8 after radiation. Anti-CD11b antibody was injected i.p. every two days starting 1 day before radiation. Anti-PD-L1 antibody was administered i.p. on day 0, 3, and 7 after radiation at 200 μg/mouse. Anti-IFN-γ was administered i.p. at 500 μg/mouse every 7 days. Anti-NK1.1 antibody was administered via i.p. at 25 μg/mouse every 3 days for 3 doses. Anti-GM-CSF antibody was given by i.p. injection at 250 μg/mouse every 2 days. FTY720 was delivered via gavage at 20 μg per dose every day for 7 days.

### Flow Cytometric Analysis

Single cell suspensions were obtained as previously described (40). Cells were blocked with anti-Fc receptor (2.4 G2, BioXCell, Lebanon, New Hampshire, United States of America) and stained for live/dead with Zombie UV, followed staining using antibodies against CD45, CD4, CD8, NK1.1, CD11b, CD11c, Ly6C, Ly6G, F4/80, CD44, CD62L.

The following antibodies were used for high-dimensional flow analysis: BV605-CX3CR, BV650-XCR1, PB-CD11 b, BV510-CD24, BV711-CD64, BV785-F4/80, AF532-CD45, PerCP-Cy5.5-1-A/I-E, PerCP-eFluor710-Flt3, PE-Cy7-CD11c, FITC-Ly6C, PE-iNOS, PE-Dazzle 594-CD103, APC-TNF, Alexa Fluor 700-CD206, and APC-Cy7-SIRPα. For IFN-γ and GrzB (515407) staining, cells were cultured with PMA/ionomycin and brefeldin A (423303, BioLegend, San Diego, California, United States of America) for 6 hours followed by intracellular staining using BD fix/perm intracellular staining kit (554714, BD Biosciences, Franklin Lakes, New Jersey, United States of America). For TNF-α staining, cells were stimulated by PMA/ionomycin and brefeldin A for 4 hours followed by intracellular staining. Foxp3 (17-5773-82, Thermo Fisher Scientific, Waltham, Massachusetts, United States of America) Ki67 (Clone 16-A8, BioLegend, San Diego, California, United States of America) and iNOS staining were performed following Foxp3 staining protocol (00-5523-00, Thermo Fisher Scientific, Waltham, Massachusetts, United States of America). Cells were analyzed on Fortessa flow cytometer (BD Biosciences, Franklin Lakes, New Jersey, United States of America) or on Aurora spectral cytometer (Cytek Biosciences, Fremont, California, United States of America. Data was analyzed with software sold under the tradename FLOWJO software (FlowJo LLC, Ashland, Oregon, United States of America).

### ELISPOT Assay for IFN-γ-Secreting CD8⁺ T Cells

MC38 cells were irradiated (40 Gy) and cultured overnight. CD8⁺ T cells were isolated with EasySep mouse CD8+ positive selection kit (18953, STEMCELL Technologies, Vancouver, Canada) from draining lymph nodes of tumor-bearing mice on day 8 after treatment. 2×10⁵ isolated CD8⁺ T cells and 2×10⁴ irradiated MC38 cells were plated onto 96-well PVDF-backed microplates coated with monoclonal antibody specific for mouse IFN-γ for 72 hours. Spots were developed by following the manufacturer's protocol (BD Biosciences, Franklin Lakes, New Jersey, United States of America).

### Generation of Bone Marrow-Derived Dendritic Cells (BMDC) and TNF-α/iNOS Producing Dendritic Cell (Tip DC) Induction In Vitro

Femurs and tibias from 8- to 10-week-old mice were harvested. BMDCs were derived as described (40). The cultured BMDC were collected on day 6 for further experiments. For Inf-MAC induction in vitro, bone marrow cells were cocultured with the presence of 200 μM RA. On day 6, indicated amount of IFN-γ was added directly to the culture. Cells were stained for iNOS and TNF-α after 24-36 hours and analyzed using flow cytometry.

### Cytokine/Chemokine and Nitrite Assay

Whole tumors were excised at indicated time point post treatment. Tumors were weighed and homogenized in PBS with the presence of protease inhibitor cocktails at a ratio of 1 ml per gram of tissue. 25 μl of supernatants were subjected to a bead-based cytokine and chemokine assay (a mouse inflammation panel and chemokine panel sold under the tradename LEGENDPLEX™ (BioLegend, San Diego, California, United States of America). The assays were analyzed using a LSRII flow cytometer and software sold under the tradename LEGENDPLEX™ (both from BioLegend, San Diego, California, United States of America). IFN-γ was measured using IFN-γ flex CBA set (BD Biosciences, Franklin Lakes, New Jersey, United States of America). TNF-α concentration was measured using ELISA kit from R&D Systems (MTA00B; Minneapolis, Minnesota, United States of America). For nitrite assay, equal weight of tumors were cut into pieces and cultured in DMEM medium for 2 hours. The production of NO in the supernatants was measured indirectly by measuring the concentration of nitrite by using the Griess reagent nitrite measurement kit (Cell Signaling Technology, Danvers, Massachusetts, United States of America; #13547).

### Immune Cell Adoptive Transfer

For T cell transfer, total T cells were isolated from lymph nodes and spleens using a T cell isolation kit (19851, STEMCELL Technologies, Vancouver, Canada). 2×10⁶ T cells were adoptively transferred into each Rag1^{-/-} mouse via retroorbital i.v. injection 2 days prior to start of the treatments. For myeloid cell transfer, bone marrow cells were harvested and washed twice with PBS. 2×10⁶ cells were transferred via retroorbital i.v. injection 1 day prior to the start of the treatments.

### RNA Extraction and Quantitative Real-Time PCR

Total RNA from tumor and CD11c+ cells isolated from tumors with a kit sold under the tradename EASYSEP® Mouse CD11c positive selection kit II (STEMCELL Technologies, Vancouver, Canada) was extracted with a kit sold under the tradename QUICK-RNA® Miniprep Plus kit (R1058, Zymo Research, Irvine, California, United States of America). cDNA was synthesized with a High Capacity cDNA Reverse Transcription kit (4368814, Applied Biosystems, Waltham, Massachusetts, United States of America). Real-time PCR was performed with a kit containing fluorescent chemicals sold under the tradename SYBR™ (Molecular Probes, Sunnyvale, California, United States of America), i.e., the SYBR™ Green PCR Master Mix (4309155, Applied Biosystems, Waltham, Massachusetts, United States of America) and results were normalized to β-actin. Primers used:

| | |
|---|---|
| iNOS | For-CACCAACAATGGCAACATCAG (SEQ ID NO: 1) |
| | Rev-GTCGATGCACAACTGGGTG (SEQ ID NO: 2) |
| TNFa | For-GCCTATGTCTCAGCCTCTTCT (SEQ ID NO: 3) |
| | Rev -TCTGGGCCATAGAACTGATGA (SEQ ID NO: 4) |
| CXCL9 | For-CGCTGTTCTTTTCCTCTTGG (SEQ ID NO: 5) |
| | Rev-AGTCCGGATCTAGGCAGGTT (SEQ ID NO: 6) |
| CXCL10 | For-CCAAGTGCTGCCGTCATTTTC (SEQ ID NO: 7) |
| | Rev-GGCTCGCAGGGATGATTTCAA (SEQ ID NO: 8) |
| CXCL11 | For-AGTAACGGCTGCGACAAAGT (SEQ ID NO: 9) |
| | Rev-GTCAGACGTTCCCAGGATGT (SEQ ID NO: 10) |
| ß-actin | For-ACAGCTTCTTTGCAGCTCCT (SEQ ID NO: 11) |
| | Rev-ATACAGCCCGGGGAGCA (SEQ ID NO: 12) |

Relative gene expression was calculated using the 2^{-ΔΔCT} approach.

### Statistical Analysis and Patient Analysis

The RSEM (RNA-seq by expectation-maximization) normalized gene expression data from TCGA PanCancer were downloaded from the cBioPortal (February 2018 version) for colorectal adenocarcinoma (COAD). For correlation analysis, a log 2 transformation was applied to the gene expression values, and then the Spearman r and P values for the correlation of all genes with NOS2 expression were computed with GraphPad Prism.

Data from animal experiments were analyzed by one-way analysis of variance (ANOVA) or two-way ANOVA with multiple comparison test or Student's t test. Kolmogorov-Smirnov test was used to test for normality. Data are presented as means±SEM. We indicated the level of statistical significance using these symbols: *P<0.05, **P<0.01, and ***P<0.001; ****P<0.0001; NS indicates no significant difference.

### Example 2

### Combining Local Ablative Ir with Atra Enhances Radiosensitivity and Antitumor Immune Memory Response

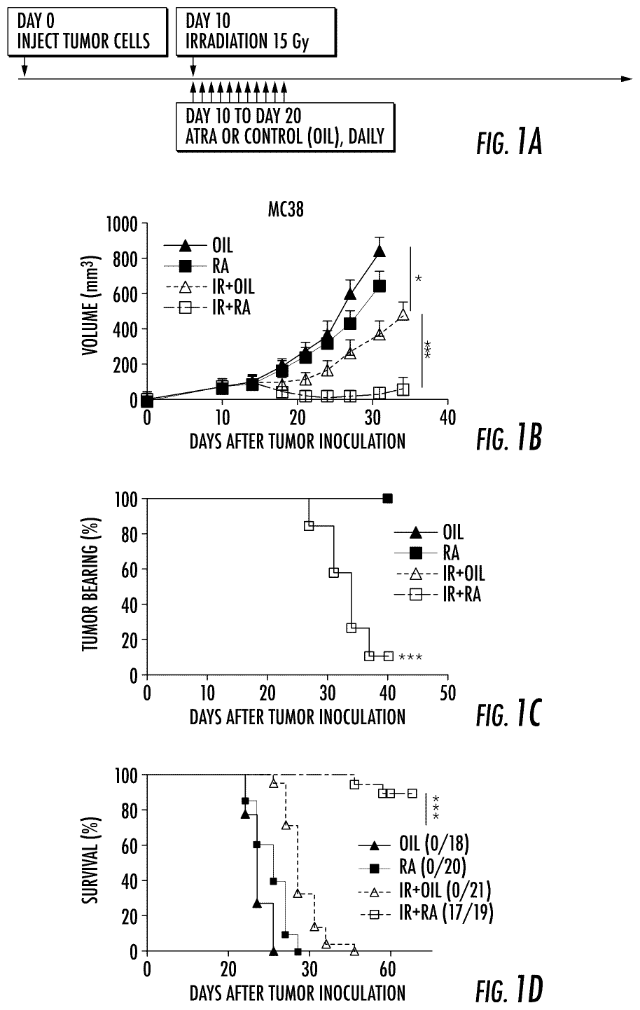

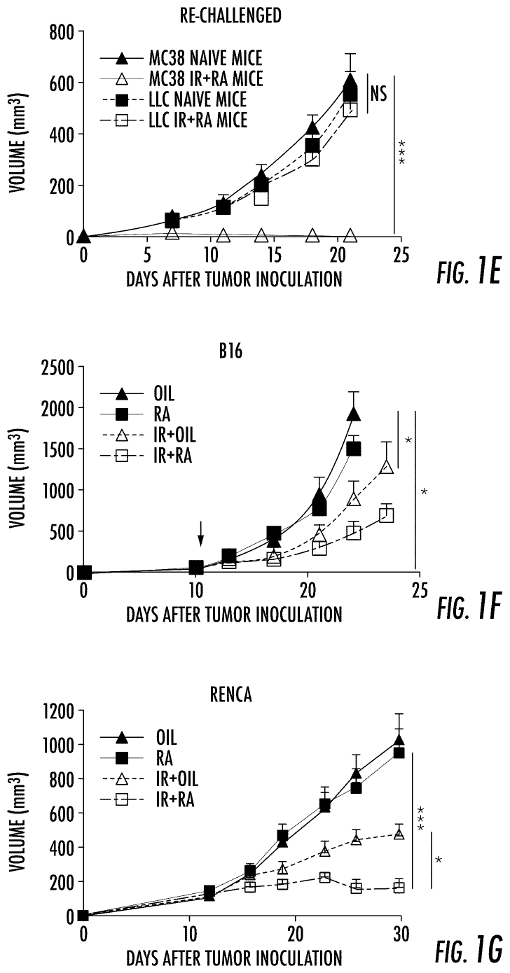

To examine the effect of the combination of local ionizing radiation with ATRA on tumor growth, we treated established syngeneic mouse MC38 colon carcinoma tumors by oral gavage with oil (control), ATRA (400 μg/dose), local IR (15 Gy), or IR plus ATRA (IR+RA). The treatment schedule is shown in FIG. 1A. IR alone inhibited tumor growth, while RA had no significant effect on MC38 tumor growth. Combination of IR with RA synergistically inhibited tumor growth compared to either the untreated controls or any single treatment. See FIG. 1B. Combination treatment resulted in complete tumor regression in 17 of 19 mice (see FIG. 1C), whereas animals that were untreated or treated by IR or RA alone did not survive more than 50 days. See FIG. 1D. The cured mice and naive mice were re-challenged with LLC cells or MC38 cells. LLC grew at the same rate in naive mice and cured mice, whereas MC38 cells did not form tumors in the cured mice. See FIG. 1E. These results suggest that the combined treatment led to tumor-specific immune memory. In the B16 melanoma tumor model, Renca (a renal cancer tumor model), and CT26 (a colon cancer model), IR and RA combination treatment also had a significant benefit compared with single treatments. See e.g., FIGS. 1F and 1G.

Significant tumor inhibition effects are also observed when combining IR with RA administered by i.p. injection, slow RA-releasing pellets implanted subcutaneously, or when RA was given at a lower dose (200 μg/dose) by oral gavage. Animals receiving higher dose RA (800 μg/dose) by gavage experienced weight loss 7-14 days post treatment, but the weight was regained by 3 weeks. However, animals that received 10 days of 400 μg/dose RA post IR did not exhibit any weight loss. If not otherwise indicated, a dose of 400 μg via gavage was used throughout the following studies. Overall, the results demonstrate that the addition of RA improved the therapeutic efficacy of ablative IR and led to a tumor-specific memory immune response.

### Example 3

### Inflammatory Macrophages and Antitumor Effect on Local IR and RA Treatment

To determine whether RA has direct cytotoxic and/or radiosensitizing effects on tumor cells, MC38 cells were cultured with RA (0 nM to 2000 nM) after IR (0 Gy to 15 Gy) for three days. RA had no direct effects on tumor cell death in vitro. Since both IR and RA regulate immune responses, studies were conducted to examine how the combination treatment changes the tumor immune environment. Using a multiplex assay, the level of chemokines and cytokines in tumors three days after treatment was determined. Following IR+RA treatment, increased levels of CCL2, CCL3, CCL4 and CCL5 (by 2-fold to 3-fold compared to levels in any single treatment), which are involved in monocyte recruitment, were observed. GM-CSF, a critical cytokine for DC generation, increased by more than 5-fold in whole tumor homogenates. Other cytokine levels, such as IFN-β, IL-6, IL-1 and IFN-γ, were also significantly elevated in IR+RA treated tumors compared to other treatments, indicating that the combination treatment of IR and RA resulted in an inflamed tumor microenvironment.

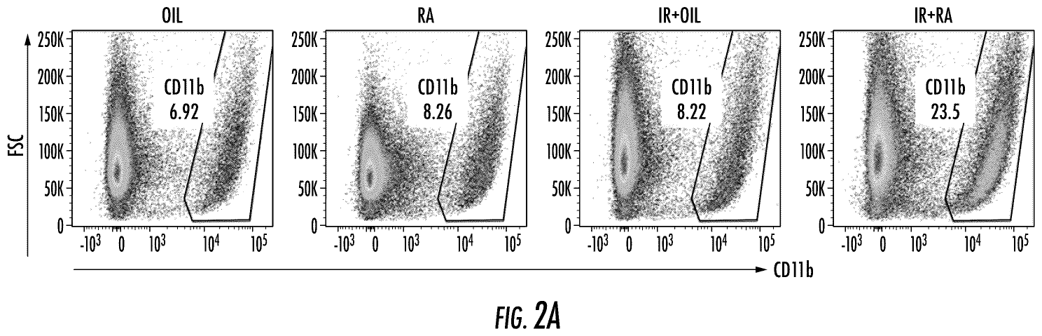

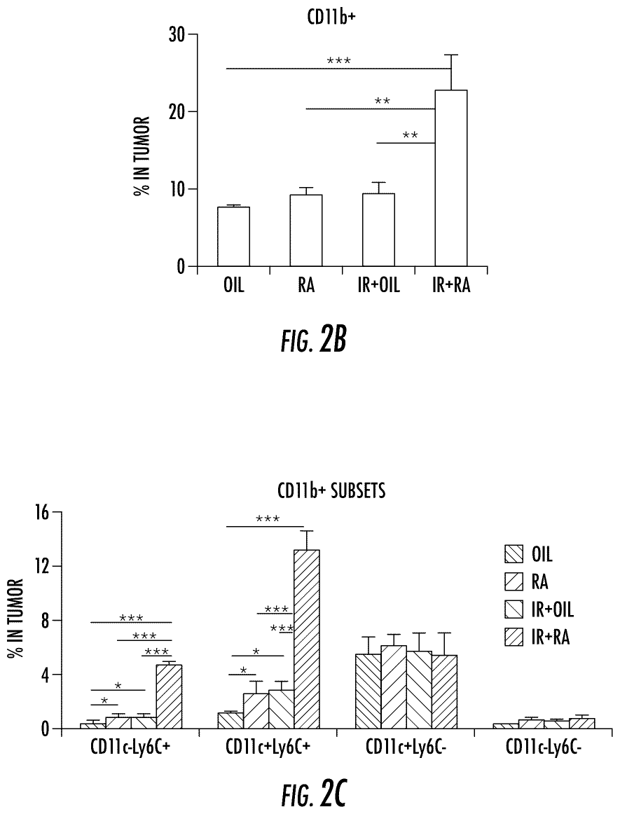

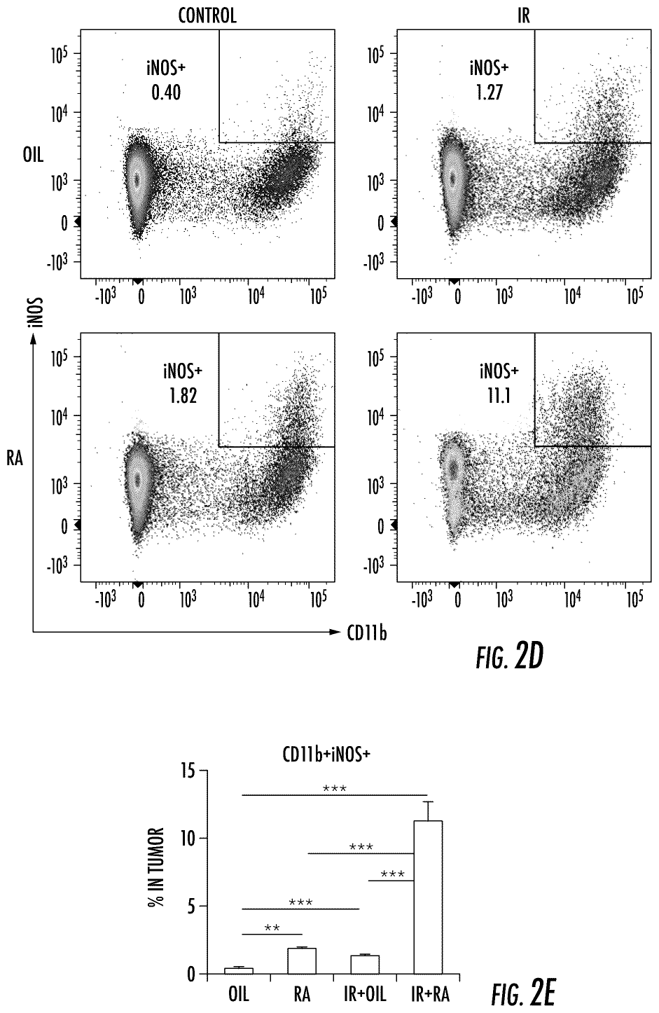

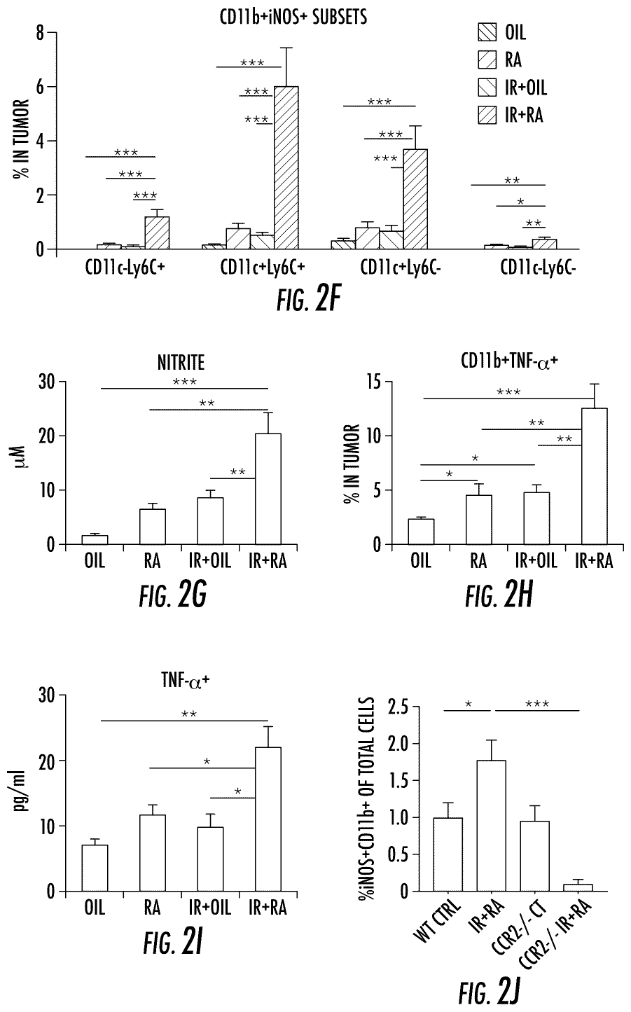

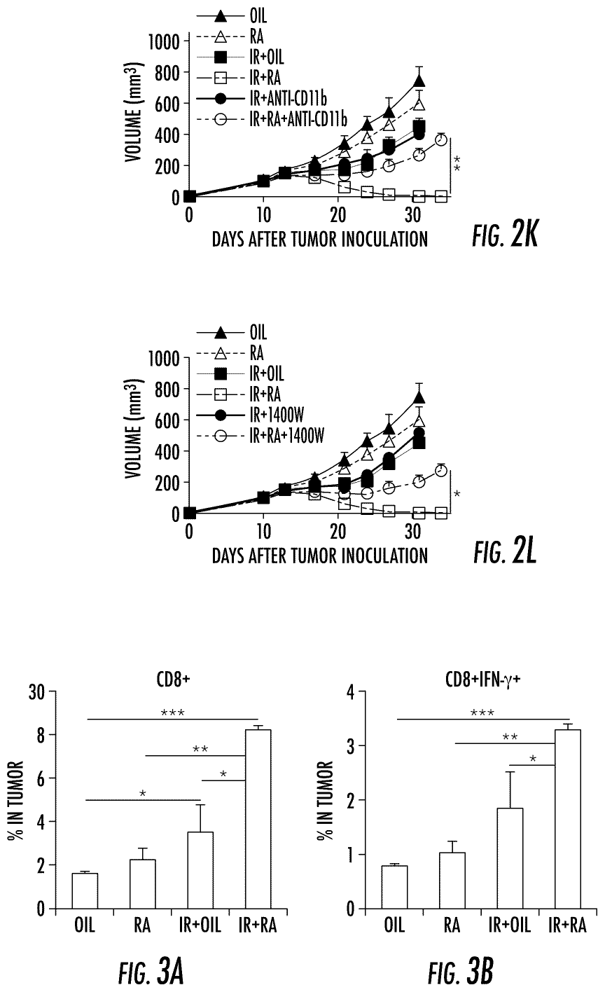

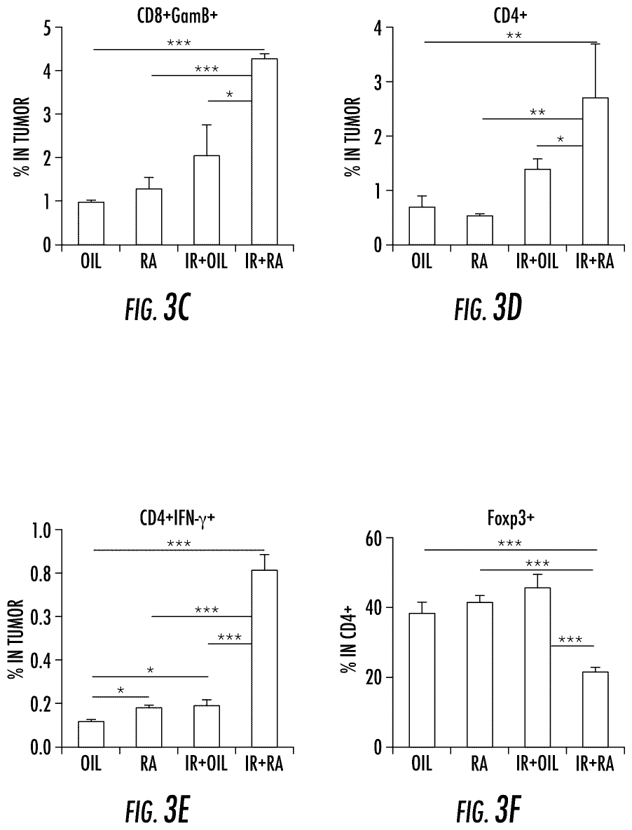

Immune cells in tumors 4 days after the start of the treatment were examined. Flow cytometry results showed that the percentage of CD45⁺ and CD11b⁺ cells increased by 2-fold to 3-fold in tumors receiving IR+RA treatment. See FIGS. 2A and 2B. In CD11b+ subsets, Ly6C⁺ cells, including CD11c⁺Ly6C⁺ cells, increased by 4-fold in tumors receiving the combination treatment. See FIG. 2C. On day 4 post treatment, there was no significant change in the percentage of CD4⁺, and there was only a marginal change in CD8⁺ T cells. Next, the DC population was examined by analyzing high-dimensional flow cytometry data using traditional dendritic cell (DC) markers. Monocyte-derived dendritic cells (MoDCs) (Ly6C⁺CD11 b⁺CD11c⁺ MHC-II⁺CXCR3⁺CD64⁺SIRPα⁺CD206⁻ cells), a low level of type two dendritic cells (DC2s) (Ly6C⁻CD11b⁺CD11c⁺MHC-11⁺CXCR3⁺CD64⁻SIRPα+CD206⁻) and a very low level of type one dendritic cells (DC1s) (Ly6C⁻CD11b⁻CD11c⁺MHC-11⁺ CXCR1⁺CD103⁺CD64⁻) were detected in IR and RA treated tumors. While the DC1 and DC2 populations were not changed during IR and RA treatment, the level of MoDCs was significantly induced.

Flow cytometry results demonstrated that the combination of RA and IR treatment dramatically increased CD11b⁺iNOS+ cells in tumors compared to any single treatment alone (0.4%, 1.8%, 1.3% and 11.1% in tumors with oil, RA, IR or IR+RA treatment, respectively). See FIGS. 2D and 2E. Overall, CD11c⁺Ly6C⁺ cells accounted for most (80%) of the increase in CD11b⁺NOS⁺ cells (see FIG. 2F) and approximately 70% of iNOS-producing myeloid cells are F4/80⁺. Since this group of cells are traditionally named TNF-α/iNOS-producing DCs (Tip-DCs), a panel of DC markers was used to further investigate this population. In the high-dimensional flow analysis employed, iNOS⁺CD11 b⁺ cells do not belong to any categories of known DCs, as most of them express CD64 and F4/80. Co-expression of CD11c, Ly6C and F4/80 in myeloid cells is one of the characteristics of inflammatory macrophages. Therefore, these cells are hereinafter referred to as “inflammatory macrophages” (Inf-MACs). A significant increase in nitrite levels was found in the culture media of IR and RA treated tumors when cultured ex vivo, suggesting an increase in nitric oxide (NO) levels in response to combination treatment (19.8 μM vs 9.5 μM, in IR+RA and IR treated tumors, respectively. See FIG. 2G. Elevated expression of TNF-α was also observed in CD11b⁺ cells after combination treatment. See FIG. 2H. Accordingly, the amount of TNF-α in tumor homogenates was significantly increased after IR and RA combination treatment (20.4 vs 11.5 μg/ml in IR+RA and RA treated tumors, respectively. See FIG. 2I. Approximately 80% of iNOS⁺myeloid cells are also TNF-α positive, while about 30% of TNF-α positive cells are double positive for iNOS. iNOS+ myeloid cells were considered to represent the majority of Inf-MAC cells. mRNA levels of iNOS and TNF-α were increased in CD11 b⁺CD11c⁺ cells sorted from treated tumors. On day 8 following the start of the treatment, more CD11b⁺CD11c⁺iNOS⁺ TNF-α⁺ cells were found in tumors treated with IR+RA compared to any other treatment group. These findings suggest that the combination treatment of IR and RA can lead to enhanced recruitment/differentiation of Inf-MACs, which can play a role for the antitumor response of IR and RA combination treatment. To verify that the source of Inf-MACs is monocytes, CCR2 knockout mice were employed in which CCR2-expressing monocytes fail to migrate out of bone marrow towards peripheral tissue, especially inflamed tissue (such as irradiated tumors). When treated with IR+RA, the number of iNOS-producing Inf-MACs was significantly lower in tumors grown in CCR2 KO hosts than tumors in WT mice. See FIG. 2J. The result indicates that monocyte infiltration during the IR/RA treatment plays a role in Inf-MAC generation.

The role of Inf-MACs and iNOS was investigated in the antitumor effect of combination treatment. CD11b⁺ cell infiltration into irradiated tumors was blocked by using an anti-CD11b antibody (33) starting on the day of treatment. The blockade was able to reduce CD11b levels after IR to the level of controls. The Inf-MAC level was also reduced during CD11 b blockade in IR+RA treated tumors. Although CD11 b blockade did not affect the efficacy of IR, the inhibition of CD11b⁺ cell recruitment into tumors significantly diminished the antitumor effect of IR and RA treatment. See FIG. 2K. When production of NO was inhibited by the iNOS specific inhibitor 1400 W in vivo, the antitumor efficacy of combined IR and RA treatment was significantly diminished. See FIG. 2L. These results indicate that Inf-MACs and iNOS induced by IR and RA treatment play a role in controlling solid tumor growth.

### Example 4

### Combining IR and RA Enhanced Tumor-Specific Adaptive Immunity in Inf-MAC-Dependent Manner

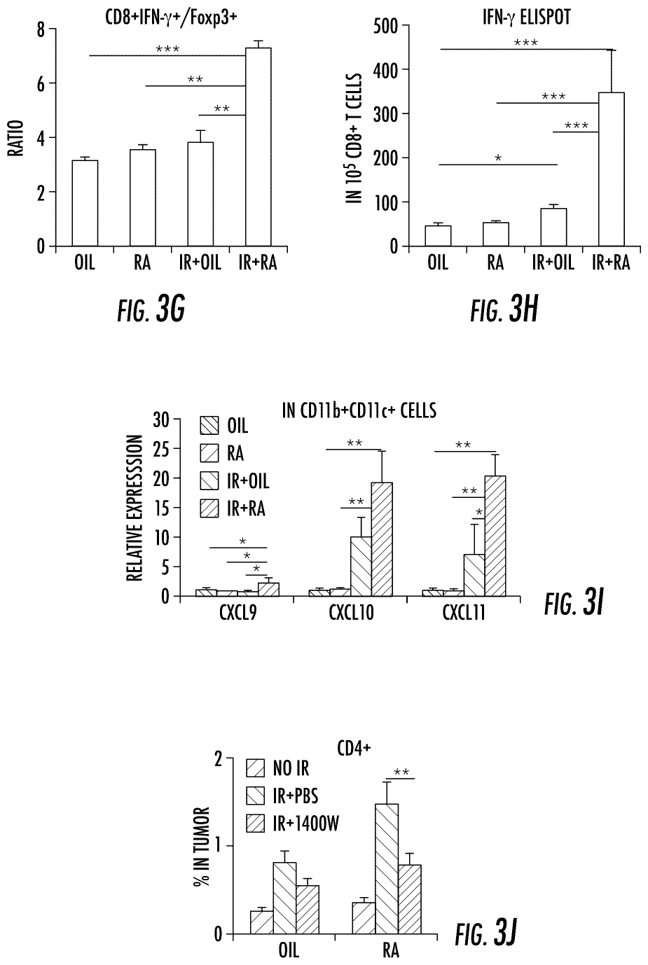

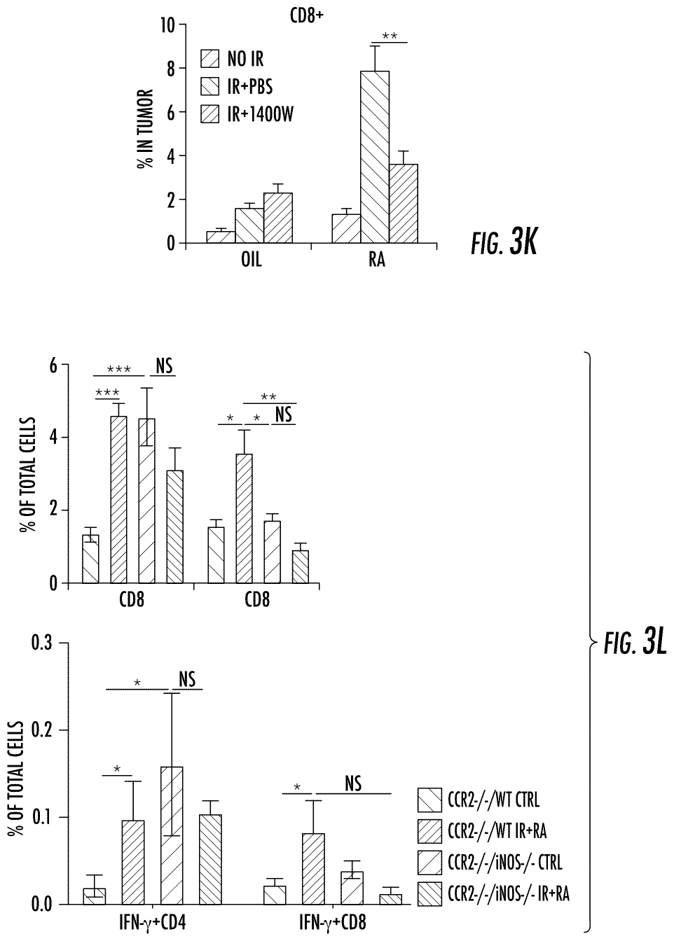

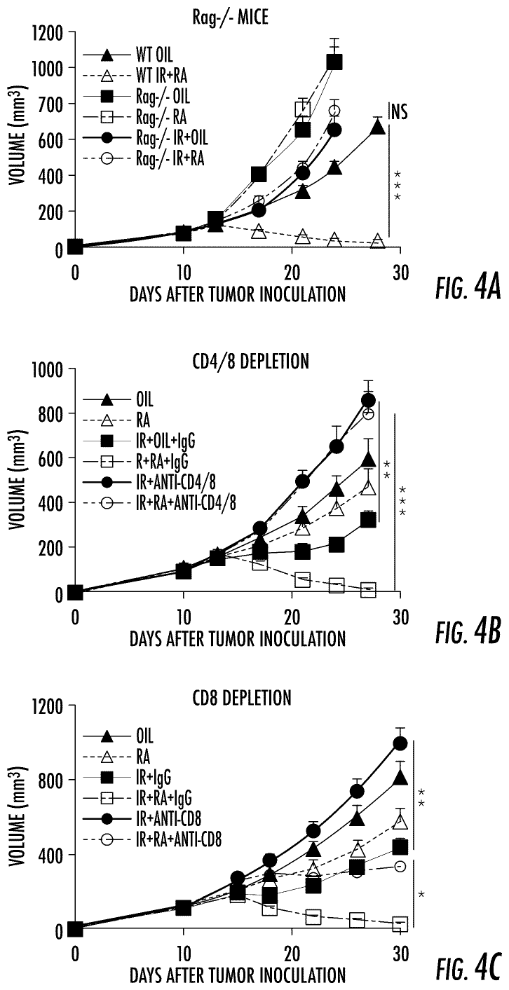

The adaptive immune response plays a role in host antitumor immunity induced by radiation (34, 35). Eight days after treatment commencement, significantly greater CD8⁺ T cells infiltration was observed in tumors receiving IR+RA combination treatment (about 3-fold increase comparing IR+RA to IR treated tumors, p<0.05), resulting in increased percentages of CD8⁺ IFN-γ⁺ and CD8⁺GzmB⁺ cells in tumors (see FIGS. 3A-3C) and draining lymph nodes (DLN) compared with those of tumors receiving single treatment. Similar increases in total CD4⁺ and CD4⁺ IFN-γ⁺ T cells were also observed in tumors (see FIGS. 3D and 3E) and DLNs. The percentage of Foxp3⁺ (T_{reg}) cells in CD4⁺ T cells decreased (see FIG. 3F), whereas the ratio of CD8⁺ IFN-γ⁺ cells to T_{reg} increased significantly in tumors which received combination treatment (see FIG. 3G) compared to those receiving any single treatment. By IFN-γ⁺ ELISPOT assay, significantly enhanced tumor antigen-specific CD8⁺ T cell priming was detected in draining lymph nodes (DLN) of mice receiving IR+RA combination treatment compared to control or single treatments alone. See FIG. 3H. In addition, the percentage of naïve T cells (CD62L⁺CD44⁻) decreased and Ki67⁺ proliferation increased in DLN for both CD8⁺ and CD4⁺ cells after the combination treatment. Without being bound to any one theory, these results suggest that IR+RA treatment enhances the activation and priming of tumor-specific T cells in tumors, and augments T cell proliferation in the DLN.

Studies were also conducted to investigate whether Inf-MACs play any role in shaping adaptive immunity. Transcription of CXCL9, CXCL10 and CXCL11, key chemokines which drive the trafficking of T cells, increased significantly in CD11c⁺ cells compared to controls. See FIG. 3I. CXCL9 and CXCL10 levels in tumors were also elevated. These results suggest that combining radiation and RA treatment can activate local adaptive immunity by enhancing T cell infiltration. Indeed, as shown in FIGS. 3J and 3K, inhibiting iNOS activity abrogated increases in the levels of CD4⁺ and CD8⁺ cells induced by IR+RA. To further investigate the relationship between iNOS-producing Inf-MACs and T cell trafficking/function, bone marrow cells (mostly CD11b⁺ cells) from WT and iNOS KO mice were adoptively transferred into tumor-bearing CCR2 KO mice in which CCR2 null monocytes are not able to traffic to inflammatory sites (tumors receiving IR+RA treatment). This transfer provides for iNOS deficient myeloid cells to populate the tumor during IR and RA treatment. Eight days after starting the IR+RA treatment, CD8⁺ and CD4⁺ T cells and their IFN-γ production were analyzed by flow cytometry. The results demonstrated that iNOS-producing Inf-MACs play a role in increased CD4⁺ and CD8⁺ T cell infiltration as well as their increased IFN-γ production during IR+RA treatment. See FIG. 3L. iNOS null myeloid cells promoted CD4⁺ T cell accumulation and their IFN-γ production in untreated tumors, compared to WT iNOS control (see FIG. 3L), which, without being bound to any one theory, is believed to be due to the defective T cell suppressive function of iNOS-null MDSCs in steady-state, compared to iNOS-WT MDSCs. These findings suggest that iNOS-producing Inf-MACs induced by IR+RA treatment in the tumor microenvironment plays a role in launching antitumor adaptive immunity.

### Example 5

### T Cells Mediate Antitumor Efficacy and Induce INF-MACS During IR and RA Combination Treatment

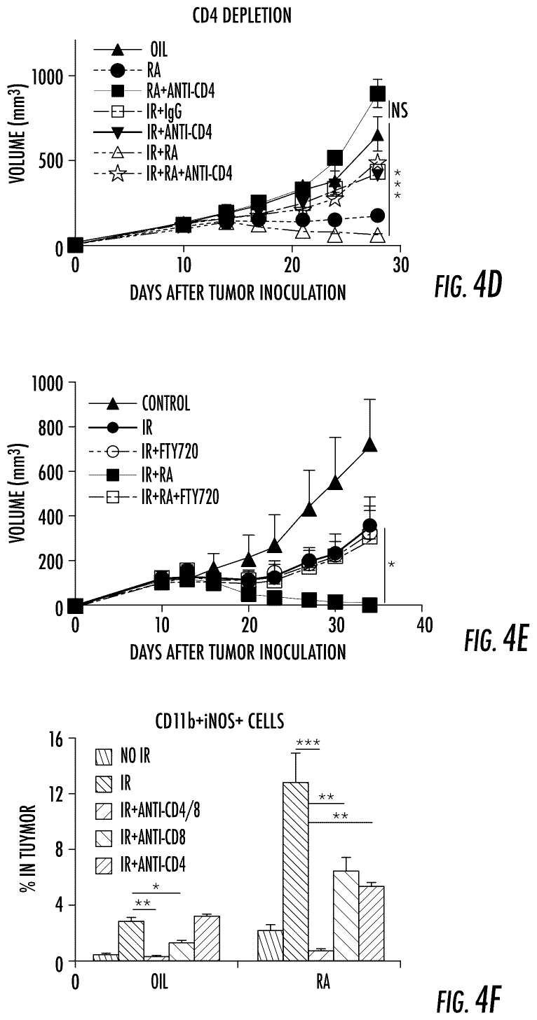

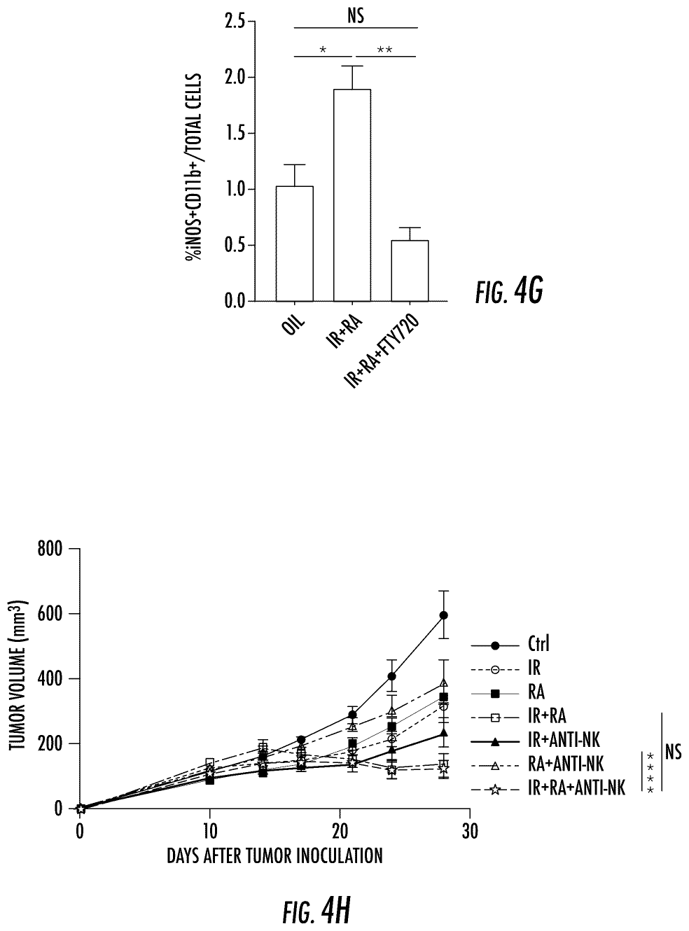

The role of T cell responses for the antitumor efficacy of IR+RA treatment was studied. Tumors grown in Rag1^{-/-} mice, which are deficient of T and B cells, did not respond to IR+RA combination treatment, in contrast to tumors in wild type mice. See FIG. 4A. To evaluate the individual contribution of CD8⁺ and CD4⁺ T cells, they were depleted in the context of IR and IR+RA treatment altogether or separately. The effect of IR alone and IR+RA treatment on tumor growth was completely abrogated by depleting both CD4⁺ and CD8⁺ T cells. See FIG. 4B. Anti-CD8 antibody completely abrogated the effect of IR alone, but only partially blocked the therapeutic effect IR+RA combination treatment. See FIG. 4C. To determine roles of CD4⁺ T cells in RA induced antitumor effect, CD4⁺ T cells were depleted in tumors treated with a higher dose of RA (800 μg/mouse/dose) for 7 days. Tumor growth was inhibited by the higher dose of RA and depletion of CD4⁺ T cells completely abolished the effect of RA. See FIG. 4D. Anti-CD4 also diminished antitumor efficacy of IR+RA treatment to the effect of either single treatment. See FIG. 4D. These results indicate that adaptive immunity, particularly the actions of CD4⁺ T cells together with CD8⁺ T cells, plays a role in the therapeutic effect of IR and RA combined treatment, during which CD4⁺ T cell function is important for RA antitumor activity. Studies were further conducted to determine whether newly infiltrated T cells play a role in the efficacy by administering the sphingosine-1-phosphate receptor agonist FTY720. FTY720 effectively blocks T cells from exiting the thymus and secondary lymphoid organs toward sites of inflammation (36). The response to IR was not affected by FTY720 treatment (37). See FIG. 4E. However, without new lymphocyte tumor infiltration, the efficacy of IR+RA treatment was diminished to the level of IR alone. See FIG. 4E. These results indicate that the superior tumor control of IR+RA treatment involves newly infiltrated T cells, which are important for the antitumor activity of RA, but not radiation. The role of T cells on Inf-MAC induction was also studied. T cell deficiency, in either Rag1^{-/-} mice or mice depleted of T cells, abrogated the iNOS expression in myeloid cells in all treatments, suggesting that T cells play a role for the induction of Inf-MACs. See FIG. 4F. More particularly, CD8⁺ T cells play a role in inducing/maintaining a high level of iNOS expression in tumors which received IR alone. CD4⁺ T cells, in addition to CD8⁺ T cells, are important for iNOS expression after RA+IR treatment. See FIG. 4F. In addition, newly infiltrated T cells play a role in a full induction of Inf-MACs in tumors treated with IR and RA as FTY720 administration abolishes increased iNOS production in Inf-MACs in tumors (see FIG. 4G) and in circulating blood after IR+RA treatment. Expression of TNF-α is also reduced when CD4⁺ and CD8⁺ T cells were depleted altogether or separately. To determine whether NK cells contribute to Inf-MAC induction and the antitumor effect of IR and RA treatment, Inf-MACs were analyzed in tumors depleted of NK cells using anti-NK1.1 antibody four days after start of the treatment. Flow cytometry showed that NK depletion did not significantly diminish the induction capacity of IR+RA. Tumor growth curves were monitored during NK depletion. The result showed that NK depletion did not alter antitumor efficacy of IR+RA treatment. See FIG. 4H. Taken together, the results reveal that T cells are not only important to mediating the antitumor activity of IR and RA treatment but are also important for inducing iNOS expression in tumor-associated myeloid cells. The results further indicate that by combining RA with IR, the antitumor power of both CD4⁺ and CD8⁺ T cells can be harnessed.

### Example 6

### IFN-γ Synergistically Promotes INF-MACS Induction by RA

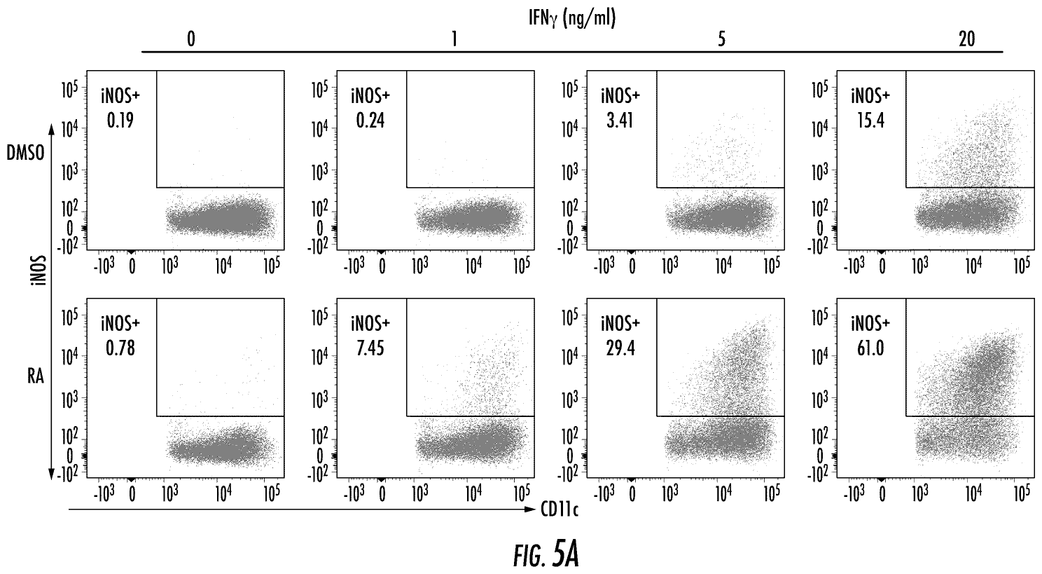

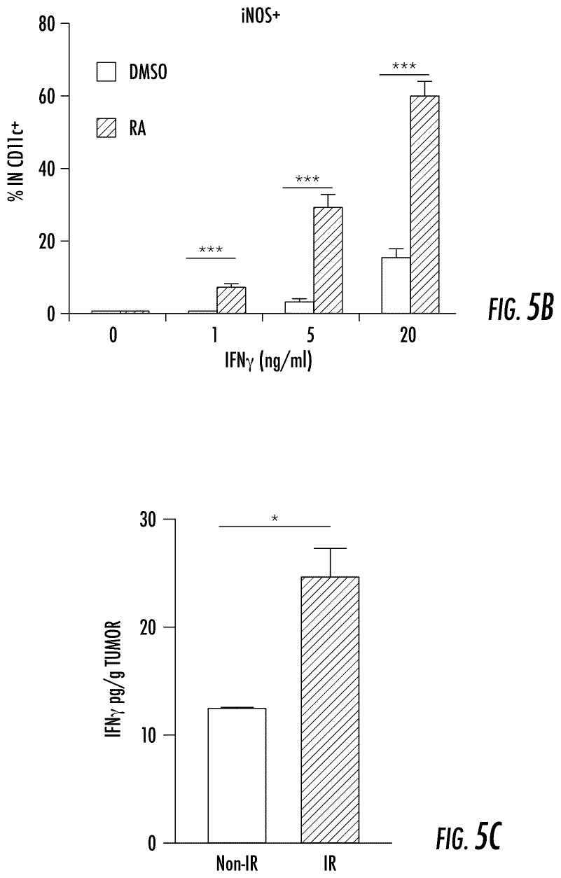

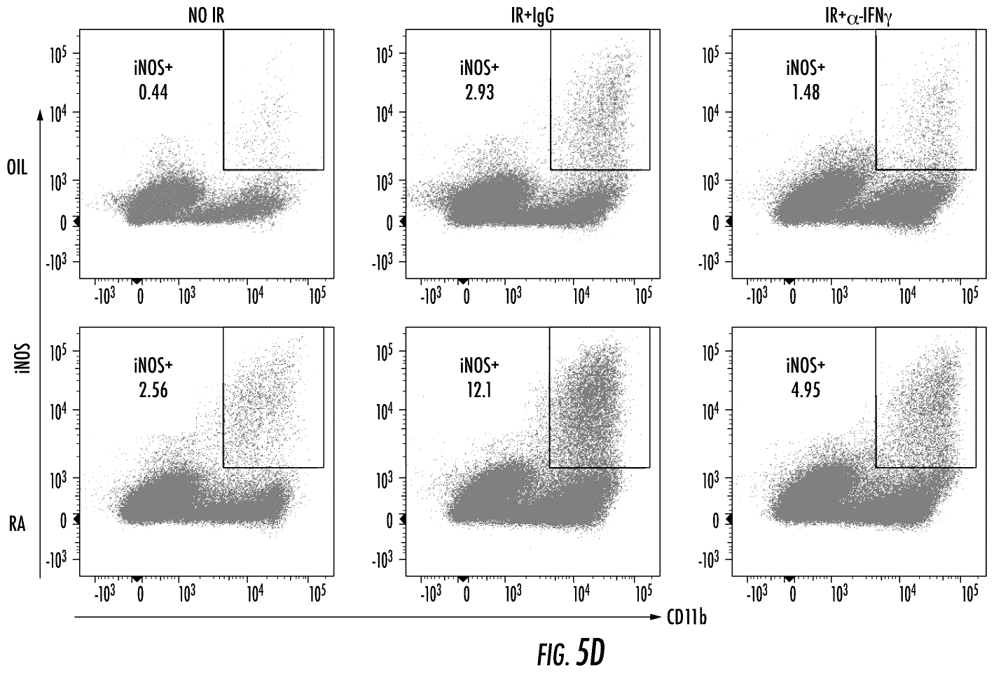

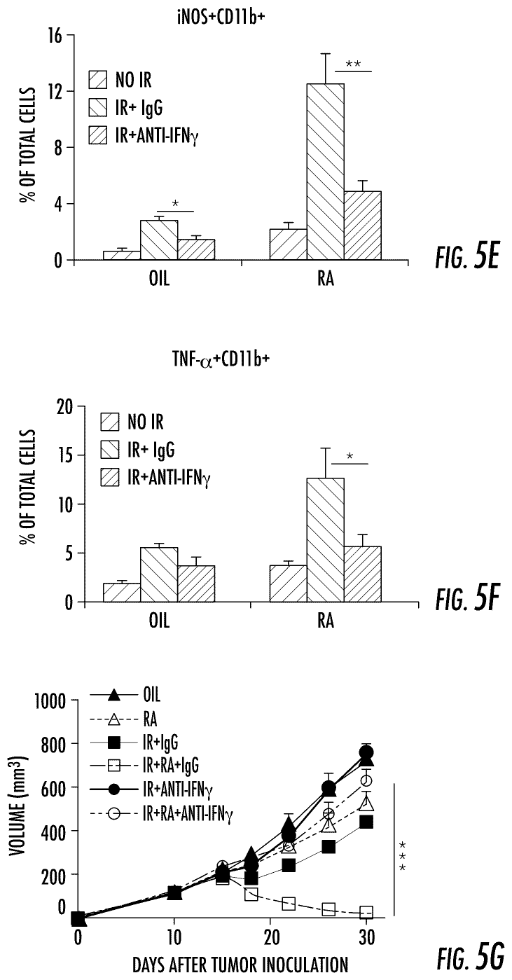

IFN-γ is a potent stimulus for iNOS expression in myeloid cells and is important for the generation of Tip-DCs in infection models (38). It was observed that iNOS production increased significantly in bone marrow-derived DCs (BMDCs) cultured in the presence of RA, and that the addition of IFN-γ synergistically increased iNOS production in a dose dependent manner. See FIGS. 5A and 5B. As described hereinabove, IR+RA treatment induced more IFN-γ⁺ T cells, and that T cells play a role in Inf-MAC generation in tumors. In addition, IFN-γ levels increased significantly by more than 2-fold in tumors treated with IR compared to untreated tumors 2 days after treatment. See FIG. 5C. When IFN-γ was neutralized in vivo using a neutralizing antibody, iNOS and TNF-α induction in myeloid cells after IR or IR plus RA treatments was significantly diminished in tumors (see FIGS. 5D-5F) and in blood PBLs. In addition, the antitumor efficacy of the combination treatment was completely abrogated by neutralizing IFN-γ. See FIG. 5G. These results suggested that IFN-γ is a main mediator of Inf-MAC induction during treatment and a major determinant of the efficacy of IR+RA. To determine whether IFN-γ produced by T cells play a role in Inf-MAC induction, T cells from WT or IFN-γ^{-/-} mice were adoptively transferred into tumor-bearing Rag1^{-/-} mice. Two days after transfer, tumors were treated by oil (vehicle) or IR plus RA. Inf-MAC induction by IR plus RA was significantly abrogated in tumors grown in IFN-γ^{-/-} Rag^{-/-} hosts compared to tumors in WT/Rag^{-/-} mice. See FIG. 5H. The result indicates that IFN-γ from T cells plays a role in Inf-MAC induction during IR and RA treatment. GM-CSF was neutralized using neutralizing antibody as the level of GM-CSF was significantly elevated in tumors treated with IR combined with RA. Inf-MAC levels during the treatment were not significantly affected by GM-CSF depletion. Taken together, these results indicate that T cell-produced IFN-γ promoted by radiation and RA converge to transform infiltrating monocytes into iNOS/TNF-α producing inflammatory macrophages.

### Example 7

### IR and RA Treatment Leads to an Abscopal Response Enhanced by PD-L1 Blockade

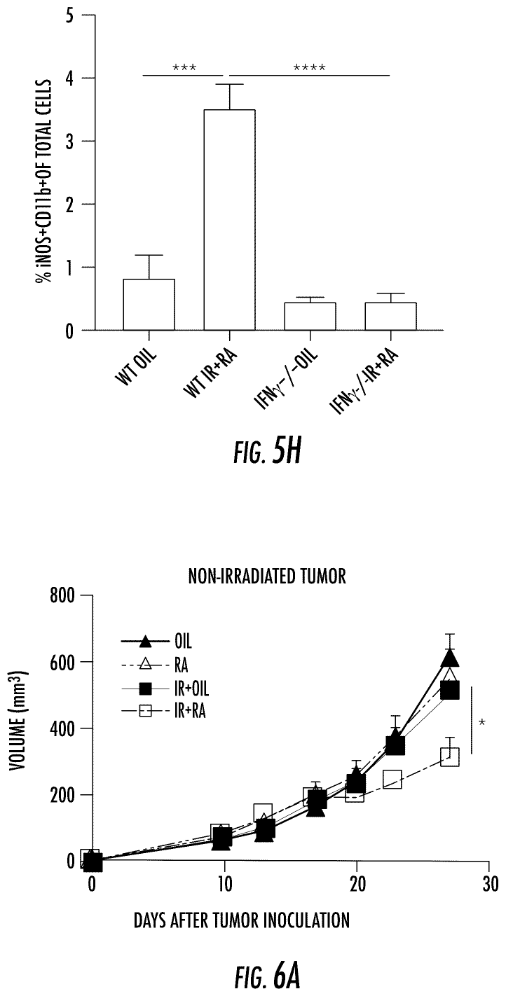

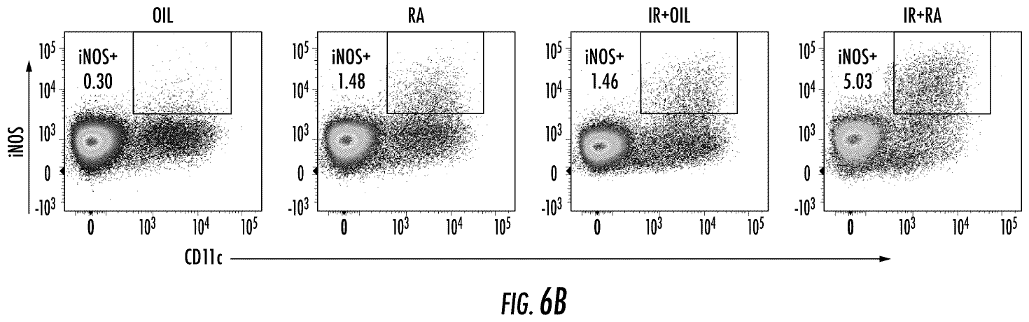

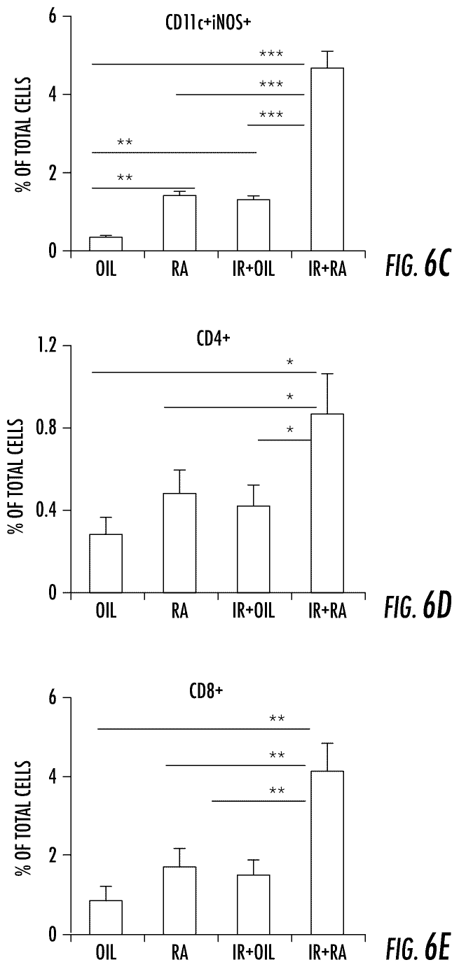

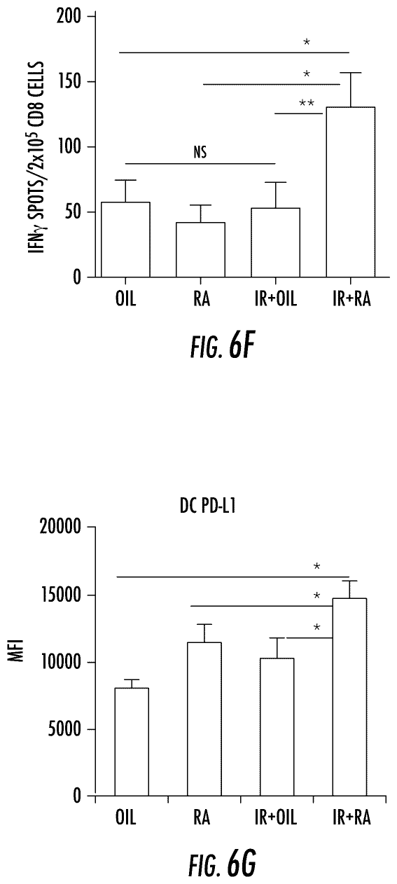

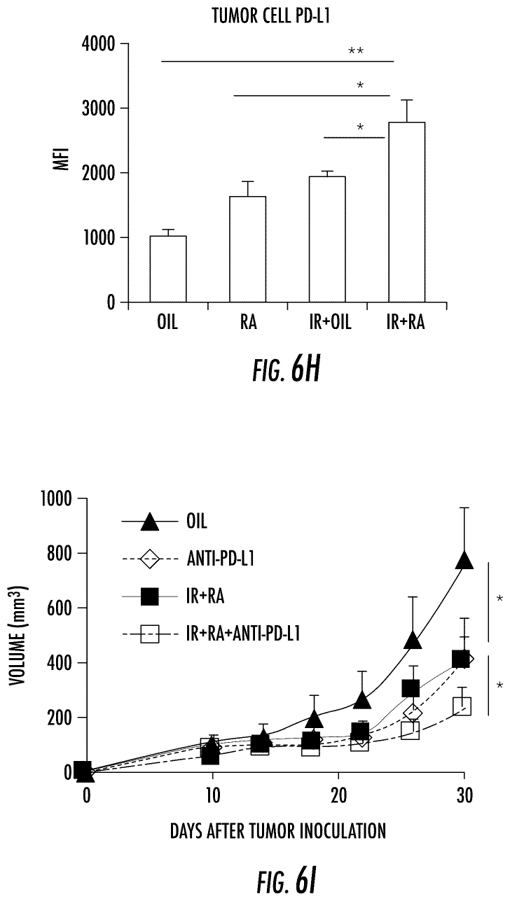

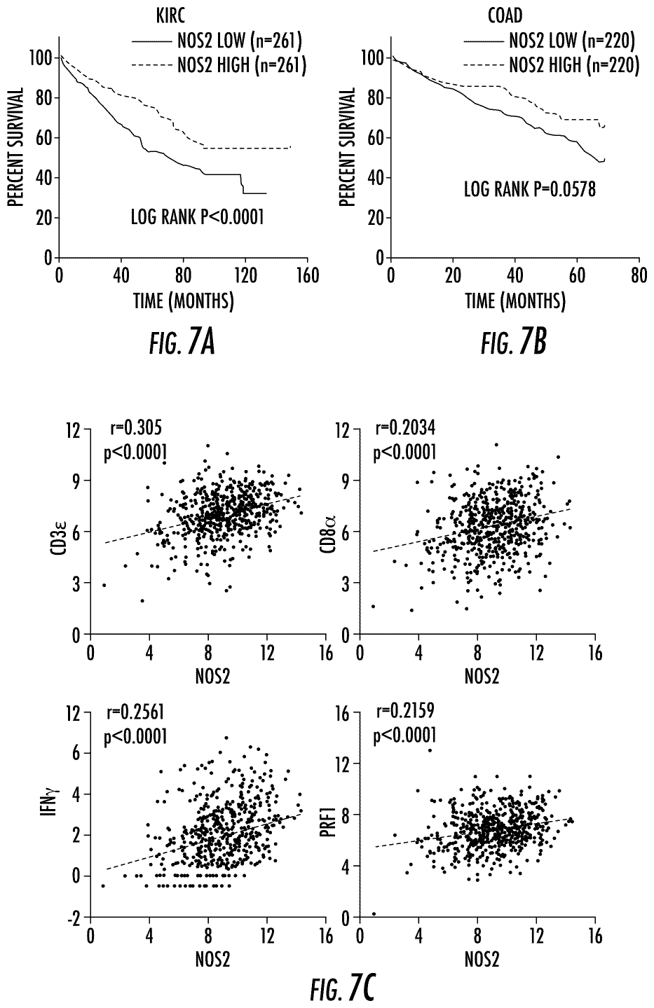

To evaluate whether the combination of IR with RA results in an abscopal effect on non-irradiated tumors, MC38 tumor cells were implanted in both flanks of mice and only the tumors on the right flank (primary) were irradiated. A slower growth rate of non-irradiated tumors was observed in the group that received RA and IR treatment compared to any single treatment alone. See FIG. 6A. This result suggests that RA not only improves the effect of IR on the primary tumor, but also enhances the systemic abscopal effect.

In the non-irradiated tumors 8 days after combination treatment, a significantly increased proportion of CD11b⁺ cells and CD11b⁺CD11c⁺Ly6C⁺ cells were observed. CD11c⁺iNOS⁺ cells also increased in the non-irradiated tumor, but not in other distal organs of the mice which received combination treatment. See FIGS. 6B and 6C. More CD8⁺ T and CD4⁺ T cells were also observed in the non-irradiated tumors when primary tumors received IR+RA treatment. See FIGS. 6D and 6E. Whereas the proportion of CD8⁺ IFN-γ⁺, CD8⁺GzmB⁺ and CD4⁺ IFN-γ⁺ cells increased within the tumor, there was a marked decrease of Foxp3⁺CD4⁺ T cells. IFN-γ production of antigen-specific T cells isolated from the DLN of non-irradiated tumors revealed that priming of CD8⁺ T cells was also augmented in the non-irradiated tumor when the primary tumor received combination treatment. See FIG. 6F. These results indicate that combination treatment of IR and RA shifted the tumor microenvironment of non-irradiated tumors towards augmented states of antitumor innate and adaptive immunity.

Increased PD-L1 expression was observed on DCs and tumor cells in non-irradiated tumors 3 days post IR+RA treatment. See FIGS. 6G and 6H. Without being bound to any one theory, it is hypothesized that employing adaptive immune checkpoint blockade by neutralizing PD-L1 could further enhance the abscopal effect of IR and RA treatment. As expected, the combination of anti-PD-L1 with IR and RA further suppressed the growth of non-irradiated tumors compared to PD-L1 blockade or IR and RA treatment alone. See FIG. 6I. These results suggest that the combination of RA and anti-PD-L1 treatment can overcome tumor radio-resistance and achieve superior control of distal tumors.

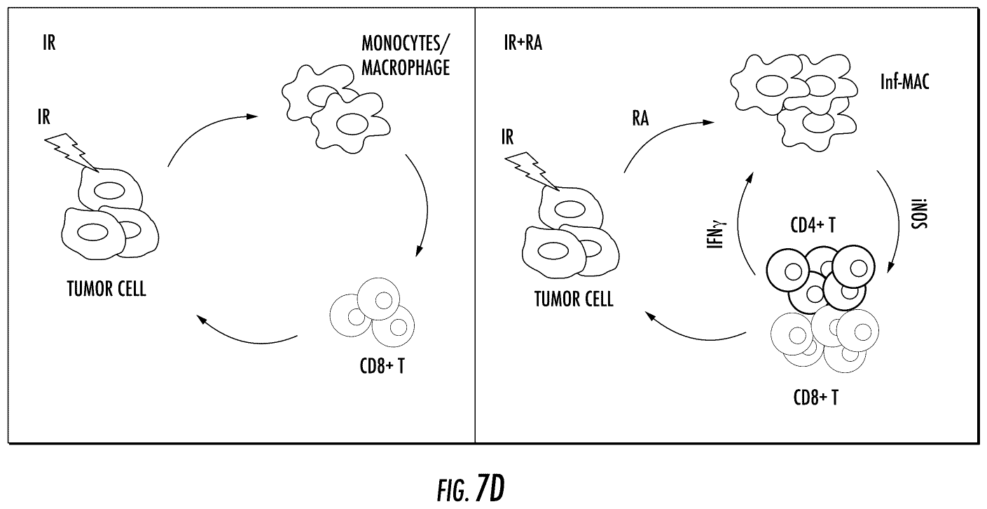

The significant correlation of iNOS expression with cancer patient prognosis is attested to by analyzing the survival of kidney and colon cancer patients in the TCGA database. Patients who had higher iNOS expression in their tumors exhibited a higher survival rate. See FIGS. 7A and 7B. Further analysis revealed that iNOS expression in a colon cancer patient cohort is significantly correlated with expression of genes related to adaptive immunity including CD3E, CD8A, IFNG and PRF1. See FIG. 7C. In summary, combining local ablative radiation and RA to treat solid tumors revealed a positive feedback loop between iNOS/TNF-α-producing inflammatory macrophages and adaptive immunity that led to local and systemic antitumor responses. See FIG. 7D. Without being bound to any one theory, it is believed that the loop starts at IFN-γ produced by tumor pre-existing T cells (37) immediately following radiation. IFN-γ, together with administered RA, drives newly infiltrated monocytes into inflammatory macrophages and further inflames the tumor microenvironment (TME). The inflamed TME calls in more T cells which in turn induces more Inf-MACs. The positive feedback loop generates inflammation and high level of T cell priming to achieve tumor local and distal control.

### Example 8

### Discussion of Examples 2-7

As described hereinabove, it appears that the combination of radiation and RA can reprogram the immune contexture of the TNE by synergistically inducing an iNOS/TNF-α-producing subset of CD11b⁺ inflammatory myeloid cells. This increase in iNOS expression mediated a dramatic increase of T cell infiltration and priming in IR+RA treated tumors. iNOS has been demonstrated to be immunosuppressive in certain contexts. In particular, NO production is a major mechanism by which MDSCs inhibit T cells. Conversely, iNOS expression is also the hallmark marker of the M1 macrophage, which can activate T cell responses. Moreover, NO is important for the differentiation of T cells (41, 42). In the instance of RA and IR combination treatment, inhibiting iNOS activity, especially in myeloid cells, diminished the T cell infiltration, IFN-γ production and antitumor efficacy effect.

As indicated in the in vitro (RA+IFN-γ) and in vivo (use of CCR2^{-/-} mice) studies described herein, Ly6C⁺CCR2⁺ monocytes are likely the main target cells of RA treatment when radiation is administered. Based on the results but without desiring to be bound by any particular theory of operation, it is proposed that tumor growth and local tumor irradiation create a highly inflammatory microenvironment that plays a role in recruitment and differentiation of inflammatory macrophages. Although these cells also circulate in blood and enter distal tumors, the local effects are much stronger. The possibility that RA can also affect intratumoral T_{reg} development cannot be discarded, as T_{reg} level also decrease in IR and RA-treated tumors. RA is reported to modulate T helper cell maturation and differentiation (45), especially by inducing Foxp3 expression. In the presently disclosed studies, it is demonstrated that when RA is combined with IR, the T_{reg} cell population did not increase and the ratio of IFNγ⁺CD8⁺ T cells to T_{reg} significantly increased. In CD4/CD8 depletion experiments, it was observed that the depletion of CD4⁺ cells diminished the antitumor effect of RA treatment and reversed iNOS induction, similar to the response to IR observed when CD8⁺ were depleted. According to the presently disclosed studies but without desiring to be bound by any particular theory of operation, it is hypothesized that therapy (RA) induced newly infiltrated CD4⁺ T cells which act as helper cells to prime CD8⁺ T cells.

The present studies demonstrate that T cells play a role in generating iNOS-expressing inflammatory myeloid cells in IR and RA treated tumors, due to production of IFN-γ, whereas inflammatory myeloid cells are important for further T cell infiltration and priming. The innate and adaptive mechanisms orchestrate a positive feedback cycle to inhibit tumor growth following IR and RA treatment. Without being bound to any one theory, it is believed that the varieties of immune cell recruitment as well as timing of activation are important for combined radio- and immunotherapy. Kidney and colon cancer patients who had higher iNOS expression exhibited prolonged survival and iNOS expression correlates with T cell adaptive immunity markers in colon cancer patients, suggesting that iNOS expression in the TME could provide a prognostic marker for immunotherapy. It is particularly noteworthy that RA, which is relatively nontoxic compared to most chemotherapies, can act as an immune modulator in reprogramming a myeloid cell-rich environment induced by IR. The presently disclosed subject matter suggests that using RA or other retinoids a non-specific T cell targeting reagents could improve the local and systemic effects of radiotherapy, thereby converting IR from a purely local modality to a systemic treatment.

### Example 9

### In Vivo Antitumor Efficacy in MC38 Tumor Bearing Mouse Models

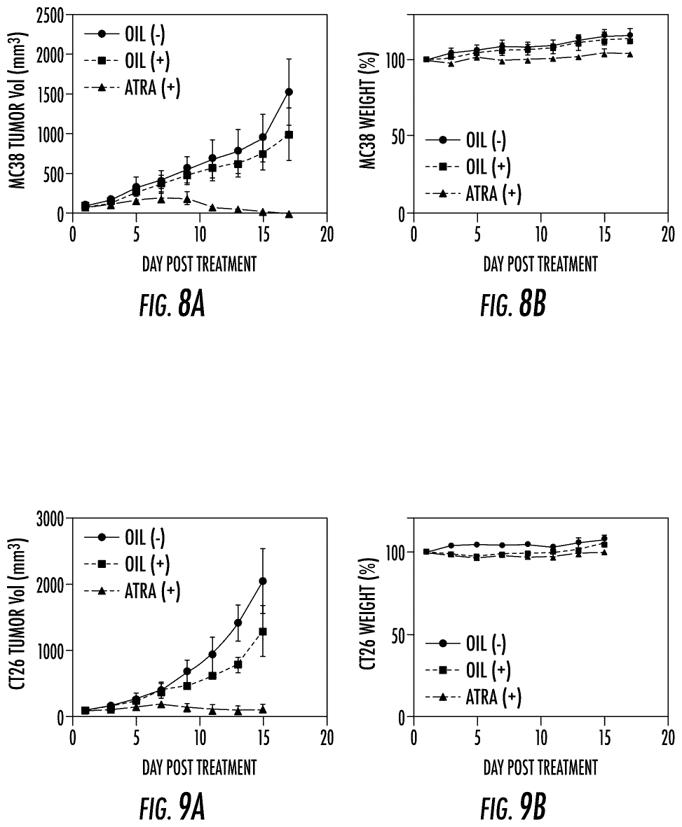

A MC38 syngeneic subcutaneous flank tumor-bearing mouse model was used for the evaluation of in vivo efficacy of ATRA-sensitized radiotherapy. 2×10⁶ MC38 cells were injected into the right flank subcutaneous tissues of C57BL/6 background mice. When the tumors reached 100-120 mm³ in volume, ATRA in corn oil (400 μg/each) was administrated via oral gavage daily. After administration, mice were anaesthetized with 2% (v/v) isoflurane and the tumors were irradiated daily with X-ray at a dose of 2 Gy/fraction (225 kVp, 13 mA, 0.3 mm-Cu filter) for a total of 3 fractions. Oil (equal volume/each) was administrated daily with or without irradiation as controls. The tumor size was measured with a caliper every day and the tumor volume calculated as 0.5×length×width². All the mice were sacrificed when the tumors reached about 2,000 mm³. The results showed that ATRA plus X-ray [denoted ATRA (+)] greatly regressed the growth of tumors. See FIG. 8A. 5 out of 5 MC38 tumor bearing mice treated with ATRA (+) were totally free of tumors, and these tumors did not grow back for 30 days, while oil with or without irradiation did not show therapeutic effects. The mice treated with ATRA and X-ray did not lose body weight (see FIG. 8B), indicating that the treatment was well tolerated by the mice.

### Example 10

### In Vivo Antitumor Efficacy in CT26 Tumor Bearing Mouse Model

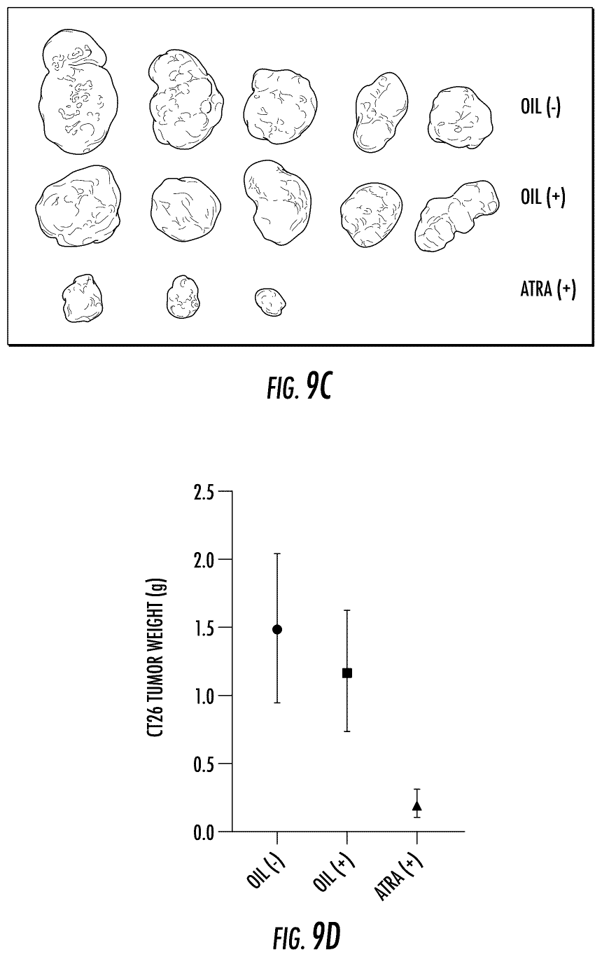

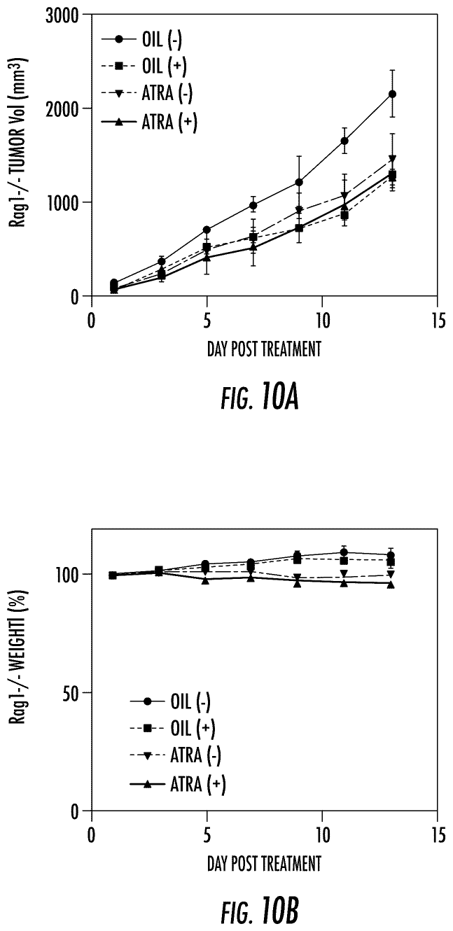

2×10⁶ CT26 cells were subcutaneously injected into the right flanks of BALB/c mice. When the tumors reached 100-120 mm³ in volume, ATRA in corn oil (400 μg/each) was daily administrated via oral gavage. After administration, mice were anaesthetized with 2% (v/v) isoflurane and the tumors were irradiated daily with X-ray at a dose of 1 Gy/fraction (225 kVp, 13 mA, 0.3 mm-Cu filter) for a total of 6 fractions on six consecutive days. Oil (equal volume/each) was daily administrated with or without irradiation as a control. The tumor size was measured with a caliper every day and the tumor volume calculated as 0.5×length×width 2. All the mice were sacrificed when the tumors reached about 2000 mm³. The results showed that ATRA (+) regressed tumor growth with 2 out of 5 mice totally free of tumors. See FIGS. 9A, 9C, and 9D. No weight loss was observed for the mice treated with ATRA (+) (see FIG. 9B), supporting that the treatment was well tolerated by the mice.

### Example 11

### In Vivo Antitumor Efficacy in MC38 Tumor Bearing Rag1−/− Mouse Model

To further study whether T cell responses are important for the antitumor efficacy of ATRA (+) treatment, 2×10⁶ MC38 cells were inoculated into the right flanks of Rag1^{-/-} 057BL/6 mice, which are deficient of T and B cells. When the tumors reached 100-120 mm³ in volume, Rag1^{-/-} mice were administered 400 ug/each ATRA daily via oral gavage followed by 2 Gy/fraction X-ray on three consecutive days. The tumor size was measured with a caliper every day and the tumor volume calculated as 0.5×length×width 2. All of the mice were sacrificed when the control group tumors reached about 2000 mm³. The results showed that ATRA (+) did not show any therapeutic effect on MC38 tumor bearing Rag1^{-/-} mouse model (see FIGS. 10A and 10B), which suggests that T cell response plays an important role hi the antitumor efficacy.

### REFERENCES

All references listed in the instant disclosure, including but not limited to all patents, patent applications and publications thereof, scientific journal articles, and database entries are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
1 M. Goldstein, M. B. Kastan, The DNA damage response: implications for tumor responses to radiation and chemotherapy. Annu Rev Med 66, 129-143 (2015).
2. S. Demaria, S. C. Formenti, Radiotherapy effects on anti-tumor immunity: implications for cancer treatment. Frontiers in oncology 3, 128 (2013).
3. R. R. Weichselbaum, H. Liang, L. Deng, Y. X. Fu, Radiotherapy and immunotherapy: a beneficial liaison? Nat Rev Clin Oncol 14, 365-379 (2017).
4. F. G. Herrera, J. Bourhis, G. Coukos, Radiotherapy combination opportunities leveraging immunity for the next oncology practice. CA Cancer J Clin 67, 65-85 (2017).
5. D. V. Krysko, A. D. Garg, A. Kaczmarek, O. Krysko, P. Agostinis, P. Vandenabeele, Immunogenic cell death and DAMPs in cancer therapy. Nat Rev Cancer 12, 860-875 (2012).
6. L. Deng, H. Liang, M. Xu, X. Yang, B. Burnette, A. Arina, X. D. Li, H. Mauceri, M. Beckett, T. Darga, X. Huang, T. F. Gajewski, Z. J. Chen, Y. X. Fu, R. R. Weichselbaum, STING-Dependent Cytosolic DNA Sensing Promotes Radiation-Induced Type I Interferon-Dependent Antitumor Immunity in Immunogenic Tumors. Immunity 41, 843-852 (2014).
7. M. M. Ahmed, J. W. Hodge, C. Guha, E. J. Bernhard, B. Vikram, C. N. Coleman, Harnessing the potential of radiation-induced immune modulation for cancer therapy. Cancer Immunol Res 1, 280-284 (2013).
8. L. Zitvogel, G. Kroemer, Subversion of anticancer immunosurveillance by radiotherapy. Nat Immunol 16, 1005-1007 (2015).
9. R. E. Vatner, S. C. Formenti, Myeloid-derived cells in tumors: effects of radiation. Semin Radiat Oncol 25, 18-27 (2015).
10. H. Liang, L. Deng, Y. Hou, X. Meng, X. Huang, E. Rao, W. Zheng, H. Mauceri, M. Mack, M. Xu, Y. X. Fu, R. R. Weichselbaum, Host STING-dependent MDSC mobilization drives extrinsic radiation resistance. Nat Commun 8, 1736 (2017).
11. J. J. Kim, I. F. Tannock, Repopulation of cancer cells during therapy: an important cause of treatment failure. Nat Rev Cancer 5, 516-525 (2005).
12. M. Diehn, M. F. Clarke, Cancer stem cells and radiotherapy: new insights into tumor radioresistance. J Natl Cancer Inst 98, 1755-1757 (2006).
13. R. A. Sharma, R. Plummer, J. K. Stock, T. A. Greenhalgh, O. Ataman, S. Kelly, R. Clay, R. A. Adams, R. D. Baird, L. Billingham, S. R. Brown, S. Buckland, H. Bulbeck, A. J. Chalmers, G. Clack, A. N. Cranston, L. Damstrup, R. Ferraldeschi, M. D. Forster, J. Golec, R. M. Hagan, E. Hall, A. R. Hanauske, K. J. Harrington, T. Haswell, M. A. Hawkins, T. Illidge, H. Jones, A. S. Kennedy, F. McDonald, T. Melcher, J. P. O'Connor, J. R. Pollard, M. P. Saunders, D. Sebag-Montefiore, M. Smitt, J. Staffurth, I. J. Stratford, S. R. Wedge, Clinical development of new drug-radiotherapy combinations. Nat Rev Clin Oncol 13, 627-642 (2016).
14. L. Altucci, H. Gronemeyer, The promise of retinoids to fight against cancer. Nature Reviews Cancer 1, 181-193 (2001).
15. X. H. Tang, L. J. Gudas, Retinoids, retinoic acid receptors, and cancer. Annu Rev Pathol 6, 345-364 (2011).
16. A. di Masi, L. Leboffe, E. De Marinis, F. Pagano, L. Cicconi, C. Rochette-Egly, F. Lo-Coco, P. Ascenzi, C. Nervi, Retinoic acid receptors: from molecular mechanisms to cancer therapy. Mol Aspects Med 41, 1-115 (2015).
17. H. de The, P. P. Pandolfi, Z. Chen, Acute Promyelocytic Leukemia: A Paradigm for Oncoprotein-Targeted Cure. Cancer Cell 32, 552-560 (2017).
18. T. Schenk, S. Stengel, A. Zelent, Unlocking the potential of retinoic acid in anticancer therapy. Brit J Cancer 111, 2039-2045 (2014).
19. A. Larange, H. Cheroutre, Retinoic Acid and Retinoic Acid Receptors as Pleiotropic Modulators of the Immune System. Annu Rev Immuno 134, 369-394 (2016).
20. M. N. Erkelens, R. E. Mebius, Retinoic Acid and Immune Homeostasis: A Balancing Act. Trends Immunol 38, 168-180 (2017).
21. J. R. Mora, M. Iwata, U. H. von Andrian, Vitamin effects on the immune system: vitamins A and D take centre stage. Nat Rev Immunol 8, 685-698 (2008).
22. Y. X. Guo, K. Pino-Lagos, C. A. Ahonen, K. A. Bennett, J. S. Wang, J. L. Napoli, R. Blomhoff, S. Sockanathan, R. A. Chandraratna, E. Dmitrovsky, M. J. Turk, R. J. Noelle, A Retinoic Acid-Rich Tumor Microenvironment Provides Clonal Survival Cues for Tumor-Specific CD8(+) T Cells. Cancer Res 72, 5230-5239 (2012).
23. N. Bhattacharya, R. Yuan, T. R. Prestwood, H. L. Penny, M. A. DiMaio, N. E. Reticker-Flynn, C. R. Krois, J. A. Kenkel, T. D. Pham, Y. Carmi, L. Tolentino, O. Choi, R. Hulett, J. Wang, D. A. Winer, J. L. Napoli, E. G. Engleman, Normalizing Microbiota-Induced Retinoic Acid Deficiency Stimulates Protective CD8(+) T Cell-Mediated Immunity in Colorectal Cancer. Immunity 45, 641-655 (2016).
24. W. Yin, Y. Song, Q. Liu, Y. Wu, R. He, Topical treatment of all-trans retinoic acid inhibits murine melanoma partly by promoting CD8(+) T-cell immunity. Immunology 152, 287-297 (2017).
25. N. Mirza, M. Fishman, I. Fricke, M. Dunn, A. M. Neuger, T. J. Frost, R. M. Lush, S. Antonia, D. I. Gabrilovich, All-trans-retinoic acid improves differentiation of myeloid cells and immune response in cancer patients. Cancer Res 66, 9299-9307 (2006).
26. S. Z. Chong, K. L. Wong, G. Lin, C. M. Yang, S. C. Wong, V. Angeli, P. A. Macary, D. M. Kemeny, Human CD8(+) T cells drive Th1 responses through the differentiation of TNF/iNOS-producing dendritic cells. Eur J Immunol 41, 1639-1651 (2011).
27. C. De Trez, S. Magez, S. Akira, B. Ryffel, Y. Carlier, E. Muraille, iNOS-producing inflammatory dendritic cells constitute the major infected cell type during the chronic Leishmania major infection phase of C57BL/6 resistant mice. PLoS Pathog 5, e1000494 (2009).
28. I. Marigo, S. Zilio, G. Desantis, B. Mlecnik, A. H. Agnellini, S. Ugel, M. S. Sasso, J. E. Qualls, F. Kratochvill, P. Zanovello, B. Molon, C. H. Ries, V. Runza, S. Hoves, A. M. Bilocq, G. Bindea, E. M. Mazza, S. Bicciato, J. Galon, P. J. Murray, V. Bronte, T Cell Cancer Therapy Requires CD40-CD40L Activation of Tumor Necrosis Factor and Inducible Nitric-Oxide-Synthase-Producing Dendritic Cells. Cancer Cell 651 (2016).
29. F. Klug, H. Prakash, P. E. Huber, T. Seibel, N. Bender, N. Halama, C. Pfirschke, R. H. Voss, C. Timke, L. Umansky, K. Klapproth, K. Schakel, N. Garbi, D. Jager, J. Weitz, H. Schmitz-Winnenthal, G. J. Hammerling, P. Beckhove, Low-dose irradiation programs macrophage differentiation to an iNOS(+)/M1 phenotype that orchestrates effective T cell immunotherapy. Cancer Cell 24, 589-602 (2013).
30. H. Xiong, S. Mittman, R. Rodriguez, M. Moskalenko, P. Pacheco-Sanchez, Y. Yang, D. Nickles, R. Cubas, Anti-PD-L1 Treatment Results in Functional Remodeling of the Macrophage Compartment. Cancer Res 79, 1493-1506 (2019).
31. S. Bhatt, J. Qin, C. Bennett, S. Qian, J. J. Fung, T. A. Hamilton, L. Lu, All-trans retinoic acid induces arginase-1 and inducible nitric oxide synthase-producing dendritic cells with T cell inhibitory function. J Immunol 192, 5098-5108 (2014).
32. Y. Devaux, S. Grosjean, C. Seguin, C. David, B. Dousset, F. Zannad, C. Meistelman, N. De Talance, P. M. Mertes, D. Ungureanu-Longrois, Retinoic acid and host-pathogen interactions: effects on inducible nitric oxide synthase in vivo. Am J Physiol Endocrinol Metab 279, E1045-1053 (2000).
33. G. O. Ahn, D. Tseng, C. H. Liao, M. J. Dorie, A. Czechowicz, J. M. Brown, Inhibition of Mac-1 (CD11b/CD18) enhances tumor response to radiation by reducing myeloid cell recruitment. Proc Natl Acad Sci USA 107, 8363-8368 (2010).
34. Y. Lee, S. L. Auh, Y. Wang, B. Burnette, Y. Wang, Y. Meng, M. Beckett, R. Sharma, R. Chin, T. Tu, R. R. Weichselbaum, Y. X. Fu, Therapeutic effects of ablative radiation on local tumor require CD8⁺ T cells: changing strategies for cancer treatment. Blood 114, 589-595 (2009).
35. H. Liang, L. Deng, S. Chmura, B. Burnette, N. Liadis, T. Darga, M. A. Beckett, M. W. Lingen, M. Witt, R. R. Weichselbaum, Y. X. Fu, Radiation-induced equilibrium is a balance between tumor cell proliferation and T cell-mediated killing. J Immunol 190, 5874-5881 (2013).
36. C. Halin, M. L. Scimone, R. Bonasio, J. M. Gauguet, T. R. Mempel, E. Quackenbush, R. L. Proia, S. Mandala, U. H. von Andrian, The S1P-analog FTY720 differentially modulates T-cell homing via HEV: T-cell-expressed S1P1 amplifies integrin activation in peripheral lymph nodes but not in Peyer patches. Blood 106, 1314-1322 (2005).
37. A. Arina, M. Beckett, C. Fernandez, W. Zheng, S. Pitroda, S. J. Chmura, J. J. Luke, M. Forde, Y. Hou, B. Burnette, H. Mauceri, I. Lowy, T. Sims, N. Khodarev, Y. X. Fu, R. R. Weichselbaum, Tumor-reprogrammed resident T cells resist radiation to control tumors. Nat Commun 10, 3959 (2019).
38. T. Bosschaerts, M. Guilliams, B. Stijlemans, Y. Morias, D. Engel, F. Tacke, M. Herin, P. De Baetselier, A. Beschin, Tip-DC development during parasitic infection is regulated by IL-10 and requires CCL2/CCR2, IFN-gamma and MyD88 signaling. PLoS Pathog 6, e1001045 (2010).
39. L. Deng, H. Liang, B. Burnette, R. R. Weicheslbaum, Y. X. Fu, Radiation and anti-PD-L1 antibody combinatorial therapy induces T cell-mediated depletion of myeloid-derived suppressor cells and tumor regression. Oncoimmunology 3, e28499 (2014).
40. Y. Hou, H. Liang, E. Rao, W. Zheng, X. Huang, L. Deng, Y. Zhang, X. Yu, M. Xu, H. Mauceri, A. Arina, R. R. Weichselbaum, Y. X. Fu, Non-canonical NF-kappaB Antagonizes STING Sensor-Mediated DNA Sensing in Radiotherapy. Immunity 49, 490-503 e494 (2018).
41. C. Bogdan, Nitric oxide synthase in innate and adaptive immunity: an update. Trends Immunol 36, 161-178 (2015).
42. Q. Xue, Y. Yan, R. Zhang, H. Xiong, Regulation of iNOS on Immune Cells and Its Role in Diseases. Int J Mol Sci 19, (2018).
43. S. Wei, S. Kozono, L. Kats, M. Nechama, W. Li, J. Guarnerio, M. Luo, M. H. You, Y. Yao, A. Kondo, H. Hu, G. Bozkurt, N. J. Moerke, S. Cao, M. Reschke, C. H. Chen, E. M. Rego, F. Lo-Coco, L. C. Cantley, T. H. Lee, H. Wu, Y. Zhang, P. P. Pandolfi, X. Z. Zhou, K. P. Lu, Active Pin1 is a key target of all-trans retinoic acid in acute promyelocytic leukemia and breast cancer. Nat Med 21, 457-466 (2015).
44. Z. Y. Wang, Z. Chen, Acute promyelocytic leukemia: from highly fatal to highly curable. Blood 111, 2505-2515 (2008).
45. J. A. Hall, J. L. Cannons, J. R. Grainger, L. M. Dos Santos, T. W. Hand, S. Naik, E. A. Wohlfert, D. B. Chou, G. Oldenhove, M. Robinson, M. E. Grigg, R. Kastenmayer, P. L. Schwartzberg, Y. Belkaid, Essential role for retinoic acid in the promotion of CD4(+) T cell effector responses via retinoic acid receptor alpha. Immunity 34, 435-447 (2011).
46. E. Alspach, D. M. Lussier, A. P. Miceli, I. Kizhvatov, M. DuPage, A. M. Luoma, W. Meng, C. F. Lichti, E. Esaulova, A. N. Vomund, D. Runci, J. P. Ward, M. M. Gubin, R. F. V. Medrano, C. D. Arthur, J. M. White, K. C. F. Sheehan, A. Chen, K. W. Wucherpfennig, T. Jacks, E. R. Unanue, M. N. Artyomov, R. D. Schreiber, MHC-II neoantigens shape tumour immunity and response to immunotherapy. Nature 574, 696-701 (2019).
47. L. F. Deng, H. Liang, M. Xu, X. M. Yang, B. Burnette, A. Arina, X. D. Li, H. Mauceri,

M. Beckett, T. Darga, X. N. Huang, T. F. Gajewski, Z. J. J. Chen, Y. X. Fu, R. R. Weichselbaum, STING-Dependent Cytosolic DNA Sensing Promotes Radiation-Induced Type I Interferon-Dependent Antitumor Immunity in Immunogenic Tumors. Immunity 41, 843-852 (2014).

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for treating a cancer in a subject in need thereof, the method comprising:
administering to the subject a retinoid or a pharmaceutically acceptable salt thereof; and
exposing at least a portion of the subject to ionizing irradiation energy.

2. The method of claim 1, wherein the retinoid is selected from the group consisting of retinol, retinal, a retinoic acid, an ester or amide of a retinoic acid, a metabolite of a retinoic acid, and mixtures thereof.

3. The method of claim 1, wherein the retinoid is selected from the group consisting of all-trans retinoic acid (ATRA), 9-cis-retinoic acid, 13-cis retinoic acid, fenretinide, retinal, 4-hydroxy-retinoic acid, 4-oxo-retinoic acid, 18-hydroxy-retinoic acid, 5,6-epoxy-retinoic acid, and mixtures thereof.

4. The method of claim 1, wherein the retinoid comprises or consists of ATRA.

5. The method of claim 1, wherein the cancer is a solid tumor cancer.

6. The method of claim 1, wherein the cancer is selected from the group consisting of a skin cancer, a connective tissue cancer, an adipose cancer, a breast cancer, a head and neck cancer, a lung cancer, a stomach cancer, a pancreatic cancer, an ovarian cancer, a cervical cancer, a uterine cancer, an anogenital cancer, a kidney cancer, a bladder cancer, a colon cancer, a prostate cancer, a central nervous system (CNS) cancer, a retinal cancer, a neuroblastoma, and a lymphoid cancer, optionally wherein the cancer is a colon cancer or a kidney cancer.

7. The method of claim 1, wherein the method further comprises administering to the subject an additional therapeutic agent or treatment.

8. The method of claim 7, wherein the additional therapeutic agent or treatment is selected from an immunotherapy agent and/or a cancer treatment, wherein the cancer treatment is selected from the group consisting of surgery, chemotherapy, toxin therapy, cryotherapy and gene therapy.

9. The method of claim 7, wherein the additional therapeutic agent or treatment comprises an immunotherapy agent.

10. The method of claim 9, wherein the immunotherapy agent is an immune checkpoint inhibitor.

11. The method of claim 10, wherein the immune checkpoint inhibitor is selected from the group consisting of a PD-1 inhibitor, a PD-L1 inhibitor, a CTLA-4 inhibitor, an IDO inhibitor, a CCR7 inhibitor, an OX40 inhibitor, a TIM3 inhibitor, and a LAG3 inhibitor, optionally wherein the immune checkpoint inhibitor is a PD-L1 inhibitor.

12. The method of claim 1, wherein the retinoid is administered orally.

13. The method of claim 1, wherein the exposing is performed by exposing said at least a portion of the subject to a fraction of a total dose of ionizing irradiation energy on two or more separate days until said at least a portion of the subject is exposed to said total dose of ionizing irradiation energy, optionally wherein said two or more separate days are two or more consecutive days.

14. The method of claim 1, wherein a combination of the administering and the exposing provides enhanced tumor growth control compared to a treatment comprising the administering alone or the exposing alone.

15. The method of claim 14, wherein the combination of the administering and the exposing provides enhanced tumor growth control for a tumor not directly targeted by said administering and/or said exposing.

16. The method of claim 1, wherein a combination of the administering and the exposing provides enhanced or comparable tumor growth control using a lower total dose of ionizing radiation energy compared to a treatment consisting of exposing the subject to ionizing radiation alone.

17. The method of claim 1, wherein a combination of the administering and the exposing provides an increase in inducible nitric oxide synthase (iNOS)-producing myeloid cells in the subject.

18. The method of claim 17, wherein the combination provides an increased level of CD11b+iNOS+ cells in a tumor in the subject.

19. The method of claim 1, wherein a combination of the administering and the exposing provides an increase in tumor necrosis factor-alpha (TNF-α)-producing myeloid cells in the subject.

20. The method of claim 1, wherein a combination of the administering and the exposing provides protection from tumor recurrence.
